(19) United States
(12) Patent Application Publication
(10) Pub. No.: US 2003/0225003 A1
(43) Pub. Date: Dec. 4, 2003

(54) Antimicrobial therapeutic compositions for oral and topical use

(76) Inventor: Dusan Ninkov, San Diego, CA (US)

Correspondence Address: O'MELVENY & MYERS LLP, 400 So. Hope Street, Los Angeles, CA, 90071-2899, US

(21) Appl. No.: 10/404,418

(22) Filed: Mar. 31, 2003

Related U.S. Application Data
(60) Provisional application No. 60/374,026, filed on Apr. 18, 2002.
(60) Provisional application No. 60/424,588, filed on Nov. 6, 2002.
(60) Provisional application No. 60/446,853, filed on Feb. 12, 2003.

(51) Int. Cl. A61K 31/7024; A61K 31/353; A61K 31/198; A61K 31/12; A61K 31/045

(52) U.S. Cl. 514/23; 514/729; 514/561; 514/456; 514/680

(57) ABSTRACT

The invention provides therapeutic antimicrobial compositions comprising natural phenolic compounds and essential oil alcohols and methods of use. The therapeutic antimicrobial compositions are useful to treat internal and external bacterial, fungal, and protozoan infections, as well as antibiotic resistant and secondary opportunistic infections.

### CROSS REFERENCE TO RELATED APPLICATIONS

This application claims priority to U.S. provisional applications Ser. No. 60/374,026 filed on Apr. 18, 2002, Ser. No. 60/424,588 filed on Nov. 6, 2002 and Ser. No. 60/446,853 filed on Feb. 12, 2003.

### BACKGROUND OF THE INVENTION

1. Field of the Invention

The present invention relates to antimicrobial therapeutic agents for oral and topical use; more specifically, the invention relates to therapeutic agents comprising combinations of carvacrol, thymol and menthol isolated from natural origins, useful in human medicine and/or veterinary medicine.

2. Description of Related Art

Treatment of infectious diseases is primarily done with antibiotics, sulfonamides, steroid hormones, antifungal, and antiprotozoan compounds. Viral infections are more difficult to treat and limited drugs are available that are effective against these diseases without significant side effects. In the case of bacterial and fungal disease, an increasing problem is the numbers of resistant organisms, some of which can be resistant to multiple drugs. These necessitate the use of antimicrobial drugs that have more toxic side effects often leading to liver and kidney damage. Thus, the development of antimicrobial compounds from natural sources that are safe, such as medicinal herbs or plants, is an advantageous solution that addresses some of these concerns. The Lamiaceae, Labiatae and Verbenaceae families of plants are known to have useful medicinal properties.

The common name for members of the Labiatae, a large family of chiefly annual or perennial herbs, is the mint family, which is classified in the division Magnoliphyta, class Magnoliopsida, order Lamiales. The mint family includes about 200 genera, such as, for example, Salvia (sage), Rosmarinus (rosemary), Mentha (mint), Ocimum (basil), Thymus (thyme), Marrubium (hoarhound), Monarda (horse mint), Trichostema (bluecuris), Teucrium, Hyptis, Physostegia, Lamium (henbit), Stachys, Scutellaria (skullcap), and Nepeta (catmint). Members of the related Verbenaceae family include Lippia (Mexican Oregano) and Lycopus. The plants in the mint family are typically shrubby or climbing, although some exist as small trees. The plants are found throughout the world.

The mint family is well known for the aromatic volatile or essential oils in the foliage, which are used in perfumes, flavorings, and medicines. Among the more important essential oils are those derived from sage, lavender, rosemary, patchouli, and the true mints. Many commonly used potherbs are from the mint family, e.g., basil, thyme, savory, marjoram, and oregano.

Numerous plant species of this family, such as catnip, pennyroyal, hyssop, self-heal, and the horehound of confectionery have a history of medicinal use in domestic remedies. Others are used as curative teas, for example, bee balm and yerba Buena.

The true mints belong to the genus Mentha. Catnip or catmint refers to a strong-scented perennial herb (Nepeta cataria) of the family Labiatae. Catnip is native to Europe and Asia and is naturalized in the United States. Although best known for its stimulating effect on cats, tea of the leaves and tops of the catnip plant have long been used as a domestic remedy for various ailments. For example, dry leaves from Nepeta cataria have been used for the production of tea, to treat restlessness, nervousness, insanity, and as a tonic for colic and carminative.

The natural compound menthol (essential oil alcohol) can be purified from the essential volatile oils of members of the Mentha genus. Menthol (1-Methyl-4-isopropyl cyclohexane-3-ol, or hexahydrothymol) is used in numerous products for its pleasing taste, aroma, and pharmacological properties. It is commonly found in pharmaceutical products, including toothpastes, mouthwashes, cough drops, and oral and nasal sprays. It is also used in confectionary foods and beverages, such as chewing gum, hard candy, chocolates, soft drinks, and liquors. It is present in many tobacco products. Perfumed lotions, after shave, shampoos, deodorants and scents often contain menthol to add aroma and enhance these products.

Menthol is known to have significant antibacterial activities and has been used in herbal treatments to cure internal and external infections. Traditional therapies have also used menthol as a mild local anesthetic. Additionally, menthol is known to have anti-spasmolytic activities and has been used as a carminative to stabilize involuntary muscle spasms of the gastrointestinal tract.

Members of the Lamiaceae, Labiatae, and Verbenaceae families of plants contain two other chemical compounds in their volatile oils that have antimicrobial activities, which are commonly referred to as carvacrol (5-isopropyl-2-methylphenol or isopropyl-o-cresol) and thymol (5-methyl-2[1-methylethyl]phenol or isopropyl-cresol). These are both monoturpene phenolic compounds and are potent antimicrobial agents. They are effective against bacteria, fungi, and protozoan pathogens. They have also been used against some parasites, such as helminths (nematodes, parasitic worms). Furthermore, it is believed that carvacrol and thymol kill these organisms by disrupting their cellular membranes and do not select resistant members of these pathogens. They are also nontoxic for animals, including humans, and have been used in a natural herb form or in oil preparations from these plants for many centuries. The development of a broad spectrum antimicrobial compound is important given the increasing problems with antibiotic resistant bacteria, resistant fungi, and resistant protozoa. The development of oral and topical antimicrobial compounds with active ingredients derived from natural sources will allow treatment of a large number of common infections caused by pathogenic microorganisms.

The antimicrobial activity of carvacrol and thymol-containing compounds is well known and has been developed for use in birds and animals. For example, U.S. Pat. No. 5,990,178 (“the '178 patent”) discloses pharmaceutical compositions containing carvacrol and thymol for treating a disease in poultry induced by hemoflagellates. Typically, carvacrol and thymol can be synthetic or obtained from the oil extract of plants such as Origanum vulgaris, Thymus vulgaris, Mentha piperita, Thymus sepilum, Saturia hortensis, Saturea montana, Saturea subricata, Carum corticum, Thymus zugus, Ocimum gratisimum, Moranda pungata, Mosla jananoica, and Salva off cinalis. The '178 patent discloses that carvacrol and thymol are effective in treating a histomoniasis protozoan infection in various birds. However, administration of monoturpene phenolic compounds is associated with irritation and pain and thus is not well suited for use in humans.

U.S. Pat. No. 6,322,825 (“the '825 patent”) discloses pharmaceutical compositions for treating gastrointestinal infections in animals. Although the '825 patent discloses that antimicrobial compounds containing carvacrol and thymol are effective against a large number of bacterial and fungal species, such compounds are likely to result in significant gastrointestinal distress in humans, thereby precluding use as human therapeutic agents.

U.S. Pat. No. 6,414,036 (“the '036 patent”) discloses antimicrobial compositions of carvacrol and thymol combined with Group I salts or organic acids, which are effective against a broad number of bacterial, fungal, and protozoan species. The '036 patent discloses compounds having enhanced antimicrobial activity due to the reaction with Group I salts or organic acids. The compounds of the '036 patent are proposed for use primarily in animal livestock, but may also have use in humans. However, the pain and discomfort associated with administration of carvacrol and thymol-containing compounds must be addressed in order to provide effective and useful human, therapeutic carvacrol and thymol-containing compounds.

Application of carvacrol and/or thymol containing compounds in human therapies must overcome several problems that are less important in veterinary therapies. One problem is the irritation and gastrointestinal (GI) discomfort and distress that may result from ingesting oral formulations of monoturpene phenolic compounds, such as carvacrol and thymol. It is desirable to have oral, topical, and injectable formulations of carvacrol and thymol for treating human and veterinary infections. One benefit of oral or topical formulations is that they allow treatment of surface skin infections, as well as internal infections, especially gastrointestinal disorders, in an easily administered manner that does not require the more advanced methods and techniques that are needed with injectable formulations. Another benefit of oral and topical formulations is avoidance of the sterility problems and dangers of contamination associated with syringes and needles needed for injections. Yet another benefit of oral and topical formulations is reduced cost of treatment.

Typically, with orally administered therapeutics the active ingredients are absorbed through the gastrointestinal tract. While with topical therapeutics the active ingredients are generally absorbed through the skin. In general oral and topical therapeutics are safe, benign, and cost-effective ways to treat infections. Accordingly, it would be desirable to provide both oral and topical formulations of carvacrol and thymol containing compounds to achieve a more cost-effective, safe, benign, and reliable treatment for internal and external infectious diseases.

### SUMMARY OF THE INVENTION

The present invention provides antimicrobial therapeutic compositions that have enhanced broad based antimicrobial activity and are effective and useful for treating infections human and animal infections. The compositions combine an organic phenolic compound with an essential oil alcohol, such as hexahydrothymol (menthol) to provide compounds that have strong antimicrobial activity with reduced gastrointestinal irritation typically associated with the administration of organic phenolic compounds. The pharmacological properties of the essential oil alcohol hexahydrothymol (menthol) provide anti-spasomolytic and anesthetic properties, thus stabilizing gastrointestinal function. The present invention also provides the essential oil alcohol hexahydrothymol and the alcohol hexahydrocarvacrol as distinct antimicrobial therapeutic agents useful alone or in combination with other antimicrobial compositions.

The organic phenolic components are typically the natural agents carvacrol and thymol that can be isolated from the volatile essential oils of plant species that are members of the Lamiaceae, Labiatae, and Verbenaceae families. The organic phenolic compound and essential oil alcohol may be combined either in a chemical reaction, or in mixtures. The volatile essential oils of some plants also contain natural tannins, natural polyphenolic molecules, and/or hypericine, all of which posses antimicrobial activities that are capable of being components of antimicrobial compositions, similar to carvacrol and thymol. Different antimicrobial formulations may be combined in mixtures of about 85-95 wt % on one to about 5-15 wt % of another to enhance activity against specific microorganisms or infection sites. In addition, the antimicrobial compositions of the present invention may be supplemented with various vitamins, minerals, amino acids, fats, and oils to aid in health and homeostasis. The antimicrobial component of the overall therapeutic compositions are usually formulated to be up to ten percent of the total weight with the remainder comprising a pharmaceutically acceptable carrier.

The antimicrobial therapeutic compositions of the present invention are useful for treating numerous bacterial, yeast, fungal, protozoan, and parasitic infections in humans and other animals. They are also useful in treating medical conditions that result from primary viral infections, stress, age, general poor health, and from a compromised immune system. They are also useful against microorganisms that have developed resistance to conventional antibiotics. The antimicrobial therapeutic compositions are intended to be administered as sterile oral and/or topical agents to treat infections. They are intended to be used primarily as human antimicrobial therapeutics. A number of different formulations may be manufactured depending on the type and location of the infection to be treated.

### BRIEF DESCRIPTION OF THE DRAWINGS

Further features and advantages of the present invention may be more readily understood by reference to the following description taken in conjunction with the accompanying drawings.

FIG. 1 illustrates the reaction of carvacrol (isopropyl-o-cresol) and thymol (isopropyl-cresol) with menthol.

FIGS. 2-1 and 2-2 illustrate an improved purification facility and method for isolating menthol, carvacrol, and thymol in highly purified forms for use in formulations of the present invention.

FIG. 3 is a graph that illustrates the percentage of people that recovered from acute diarrhea after treatment with an oral preferred formulation of the present invention (Experimental Group 1). A total of 47 people were treated in group 1. Treatment consisted of two 500 mg capsules every 12 h.

FIG. 4 is a graph that illustrates the percentage of people that recovered from traveling diarrhea after treatment with an oral preferred formulation of the present invention (Experimental Group 2). A total of 23 people were treated in group 2. Treatment consisted of two 500 mg capsules every 12 h.

FIG. 5 is a graph that illustrates the percentage of people that recovered from subacute diarrhea after treatment with an oral preferred formulation of the present invention (Experimental Group 3). A total of eight people were treated in group 3. Treatment consisted of two 500 mg capsules every 8 h.

FIG. 6 is a graph that illustrates the percentage of people that recovered from chronic diarrhea after treatment with an oral preferred formulation of the present invention (Experimental Group 4). A total of five people were treated in group 4. Treatment consisted of two 500 mg capsules every 8 h.

FIG. 7 is a graph that illustrates the percentage of people that recovered from food poisoning diarrhea after treatment with an oral preferred formulation of the present invention (Experimental Group 5). A total of three people were treated in group 5. Treatment consisted of two 500 mg capsules every 8 h.

FIG. 8 illustrates the synthesis of menthocarvol (hexahydrocarvacrol) via the reduction of carvacrol in a nitrogen atmosphere.

FIG. 9 illustrates the novel compound menthocarvol as a distinct entity via GMC chromatography. Menthocarvol is compared to carvacrol from which it was synthesized. Menthocarvol is represented by gradient 90 (133) and the starting material carvacrol is represented by gradient 92 (135).

FIG. 10 illustrates the novel compound menthocarvol as a distinct entity via GMC chromatography. The peak gradient 100 (19.98) represents menthocarvol.

### DETAILED DESCRIPTION OF PREFERRED EMBODIMENTS

The following description is of the best mode presently contemplated for practicing the invention. This description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout.

The invention provides therapeutic pharmaceutical compositions that include an essential oil extract from plants of the Lamiaceae, Labiatae, and Verbenaceae families. In particular, the antimicrobial pharmaceutical compositions include an organic phenolic compound, such as carvacrol (isopropyl-o-cresol or 5-isopropyl-2-methylphenol) and/or thymol (isopropyl-cresol or (5methyl-2[1-methylethyl]phenol). The related essential oil alcohol compound of menthol (1-Methyl-4-isopropyl cyclohexane-3-ol, or hexahydrothymol) can also be purified from the Lamiaceae, Labiatae and Verbenaceae families, preferably from members of the genus Mentha and more preferably from the species Mentha piperita. The organic phenolic or alcohol compounds are ideally obtained from plant oil extracts, but can also be synthesized by known methods. In one embodiment, the organic phenolic compounds carvacrol and/or thymol are combined with the natural antibacterial and anti-spasmolytic agent, menthol, through a chemical reaction to form a novel compound. In another embodiment carvacrol can be hydrogenated to form hexahydrocarvacrol. As used herein, the term “antimicrobial compound” refers to unreacted carvacrol, thymol, menthol, hypericine, natural tannins, natural polyphenols, and combinations thereof. The term also refers to menthol reacted carvacrol, menthol reacted thymol, menthol reacted hypericine, menthol reacted natural tannins, and menthol reacted natural polyphenols, and combinations thereof. In addition as used herein, the term “antimicrobial compound” also refers to hexahydrocarvacrol, as well as the reduced forms of hypericine, natural tannins, natural polyphenols, and combinations thereof.

The therapeutic pharmaceutical compositions of the present invention are suitable for treating humans, or other animal subjects, with internal or external microbial infections or inflammation conditions, particularly gastrointestinal infections, as well as surface infections of the skin. Animals that may be treated with the compositions of the present invention include, but are not limited to, humans, apes, monkeys; horses, cows, pigs, sheep, goats, rabbits; dogs, cats, birds, chickens, turkeys, ducks, and the like.

Because the antimicrobial compounds are degraded by enzymes, the therapeutic pharmaceutical compositions of the present invention are particularly well suited for treating microbial infections. Organic phenolic compounds such as carvacrol and thymol are degraded by enzymes into inactive metabolites, which can be excreted in the urine or expired from the lungs in the form of carbon dioxide. Mechanisms of degredation of carvacrol and thymol are well known in the art. Additionally, antimicrobial compounds of the invention do not appear to be mutagenic or carcinogenic.

Furthermore, the compounds of the present invention do not select for pathogen resistance because the compounds of the present invention putatively destroy the cell membranes of the pathogenic microorganism thereby causing its death. As used herein, the term “antimicrobial activity” includes bactericidal, fungicidal, protozoanicidal, and other disinfective activity.

I. Antimicrobial Organic Phenolic Compounds

The antimicrobial compounds of the present invention include organic phenolic compounds, such as carvacrol (isopropyl-o-cresol; or 5-isopropyl-2-methylphenol) or thymol (isopropyl-cresol, or 5-methyl-2[1-methylethyl]phenol) or the related compound of menthol (1-Methyl-4-isopropyl cyclohexane-3-ol, or hexahydrothymol). Carvacrol (isopropyl-o-cresol) is a crystal having a boiling point of about 233° C. at atmospheric pressure. Thymol (isopropyl-cresol) is a liquid that has a boiling point at atmospheric pressure of 237-238° C. Both compounds volatilize in water vapor. Menthol is an essential oil alcohol that is a colorless crystal with a pleasing mint odor that has a boiling point of about 212° C. at atmospheric pressure. In one preferred embodiment of the present invention, the antimicrobial compound is an organic phenolic compound, such as carvacrol or thymol, combined with the natural compound menthol. In another preferred embodiment carvacrol is converted into hexahydrocarvacrol by reduction of its double bonded phenolic ring structure and is referred to herein, as menthocarvol.

Organic phenolic compounds, such as carvacrol, thymol, and the related compound menthol, can ideally be isolated and purified from plant essential oil extracts, or also made synthetically by known methods. Preferably, the oil is extracted from a member of the Lamiaceae, Labiatae or Verbenaceae family. The Labiatae family includes about 200 genera, including Salvia, Rosmarinus, Mentha, Ocimum, Thymus, Marrubium, Monarda, Trichostema, Teucrium, Hyptis, Physostegia, Lamium, Stachys, Scutellaria and Lycopus. Suitable plants from which organic phenolic compounds may be extracted include, but are not limited to, Ocimum spp., Saturea spp., Monarda spp., Origanum spp., Thymus spp., Mentha spp., Nepeta spp., Teucrium gnaphalodes, Teucrium polium, Teucrim divaricatum, Teucrim kotschyanum, Micromeria myrifolia, Calamintha nepeta, Rosmarinus officinalis, Myrtus communis, Acinos suaveolens, Dictamnus albus, Micromeria fruticosa, Cunila origanoides, Mosla Japonoica, Maxymowitz, Pycnanthemum nudum, Micromeria Juliana, Piper betel, Trachyspermum antmi, Lippia graveolens and the like. In one preferred embodiment, carvacrol and thymol containing oils are purified from the species Origanum vulgare of a hirtum variety. Preferably, this is a hybrid strain that produces high quality oils. In another preferred embodiment, the oil is extracted from a plant of the genus Nepeta, such as Nepeta racemosa (catmint), Nepeta citriodora, Nepeta elliptica, Nepeta hindostoma, Nepeta lanceolata, Nepeta leucophylla, Nepeta longiobracteata, Nepeta mussinii, Nepeta nepetella, Nepeta sibthorpii, Nepeta subsessilis and Nepeta tuberosa. In another preferred embodiment, menthol oil is extracted preferably from the species Mentha piperita, but can be also be preferably isolated from other members of the Mentha genus.

Organic phenolic compounds, such as carvacrol, thymol, and the alcohol menthol (hexahydrothymol) are soluble in lipids. Such lipid solubility may play a role in the antimicrobial activity of the organic phenolic compounds of the present invention.

II. Extraction of Carvacrol and Thymol from Plants

A. Cultivating the Plant

Plants of the Lamiaceae, Labiatae and Verbenacea families are found throughout the world and are relatively easy to cultivate. To cultivate the plants, seeds, preferably those with a high percentage of phenolic compounds, are planted in fine loose soil, preferably in a sub-tropical climate (containing about 70 wt % to about 80 wt %, of organic phenolic compound). Hybrid seeds having a high percentage of organic phenolic compounds can be produced by known techniques. The hybrid seeds may then be cultivated using known agricultural techniques, such as watering and artificial fertilizing.

Because the leaves contain a high amount of oil upon blossoming, it is preferred that the plants be harvested soon after the plants begin to blossom. Preferably, the plants are harvested within 24 hours after blossoming, and more preferably within 12 hours after blossoming. Most preferably, harvesting is undertaken early in the morning or late in the evening hours when the leaves are not exposed to the sun. Because the majority of the oil is found in the leaves and blossoms of the plant, it is preferred that the leaves and blossoms be utilized in the extraction process. Use of other parts of the plant may increase impurities and decrease yield.

B. Extracting Oil from the Plant

Oil-containing organic phenolic compounds can be extracted from either dried or fresh plants, or both. If the plant is dried, the drying process is preferably undertaken in drying houses that are constructed to allow constant, free circulation of air. Preferably, the harvested leaves and blossoms should not be exposed to direct sunlight, because exposure to sunlight may reduce the amount of active material present in the leaves.

To begin the drying process, the leaves and blossoms typically are arranged in layers of 20-25 cm thick. To promote uniform drying, the layers should be turned up-side-down either manually or mechanically daily, preferably more than once a day, more preferably multiple times a day (e.g. four times a day). This rotation takes place preferably during the first few days of drying, typically within the first three days. Generally, the leaves are dried for about 7 to 8 days.

After the leaves and blossoms are dried, the oil can be extracted by known methods. The development of new improved purification methods allows isolation of carvacrol (isopropyl-o-cresol), thymol (isopropyl-cresol) and menthol (hexahydrothymol) at purity levels in excess of 98-99%.

C. Purification Facility: Coupled vacuum distillation and fractionation

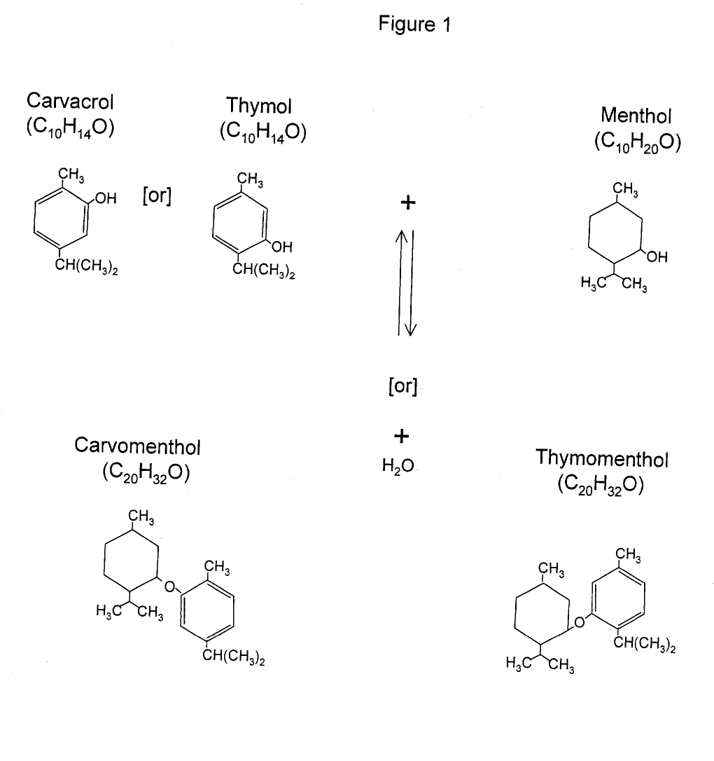

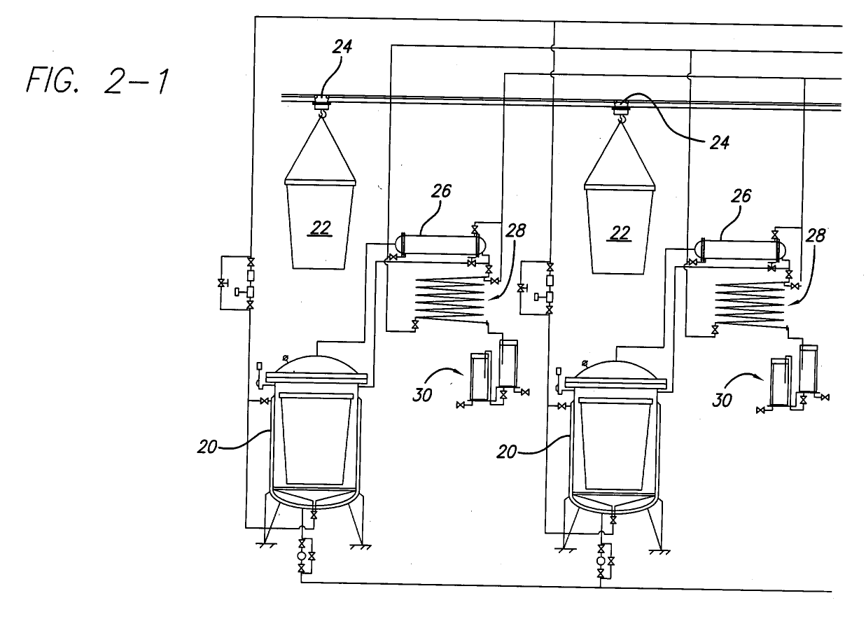

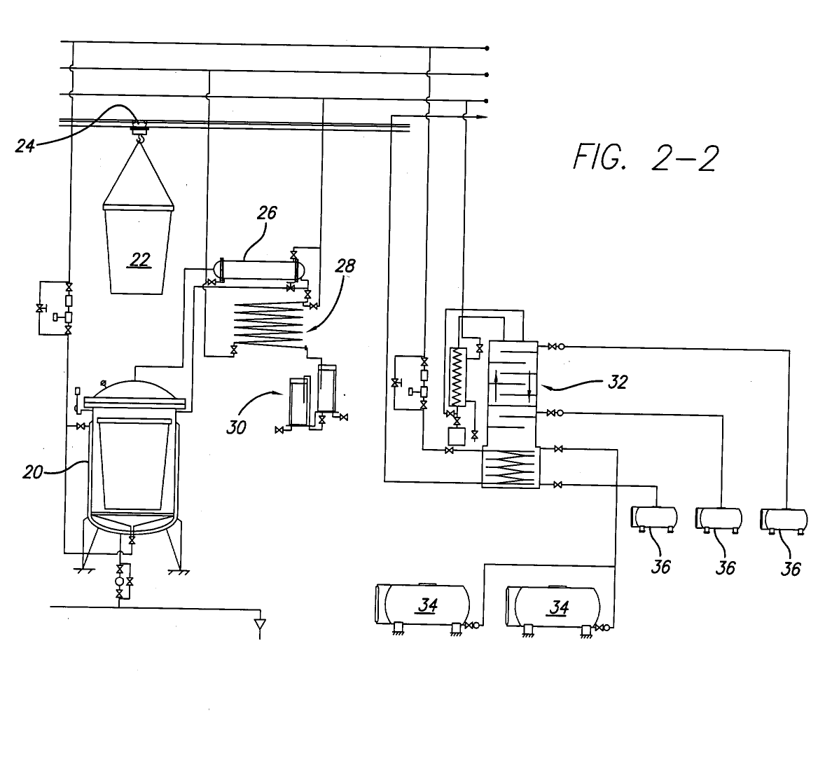

FIG. 2 illustrates an improved purification facility in which vacuum distillation and fractionation processes are coupled thereby ensuring high quality oils and increased purity and yield of the final products (FIGS. 2-1, 2-2). The improvements also result in increased efficiency and cost savings.

In order to hasten the distillation process, the herb/plant material is fragmented in stainless steel units (not depicted) fitted with stainless steel blades. The herb/plant components are ideally fragmented about 15-20 minutes prior to the start of distillation, which timing facilitates preservation of the highly volatile oils. Leaves, stems, flowers, roots, and the fruit from fresh or dried plants can be distilled in the production facility.

As shown in FIG. 2, the herb/plant material is placed into stainless steel buckets 22 having perforated bottoms. The central part of the bucket 22 is approximately 0.2 m in diameter and approximately 1 m long, allowing for easier passage of water vapor and facilitates connection of the bucket/cradle 22 to the c22olumn 32 in the distillation unit 20. A self propelled overhead crane 24 is mounted above a bank of three distillation units 20 for transporting of the fragmented herb/plant material in the buckets 22 to the individual distillation units 20. Each distillation unit 20 has the capacity to handle approximately 400 to 900 kilograms (kg) of material and can withstand a vacuum rating of about 0.9 bar or absolute pressure rating of about 0.1 bar. The distillation units 20 are fitted with hydraulic lids and easy opening fly bolts for ease of use. The pipe connection to the distiller from the top includes a joint and/or elbow to allow for movement as the pipe is released from its lid. This is a fast detachable connection. Undoing the fly nuts allows removal of the pipe connection and activation of the hydraulic (opening or closing) of the lid. The presence of two buckets 22 per each distillation unit 20 increases the efficiency of the process.

A steam generator (not depicted) ideally provides at least 1000 kg per hour (h) of steam suitable for purification of carvacrol, thymol and menthol products. The steam will be transported through receiving groups to the distilling units 20 at controlled temperature and pressure. It is important to maintain temperature regulation as the essential oils to be isolated are not stable at high temperatures. Oxidation may take place when temperatures are greater than 50° C. The vacuum distillation method employed avoids maceration of the oils at high temperatures. One unique feature of the facility is the way in which vacuum is provided in the distillation process. A compressor (not depicted) is used to provide the required vacuum. The compressor is simply fitted to the part of the piping connecting the distillation unit 20 without the need for vacuum pumps that frequently result in large losses of the precious oil products. The present method of creating vacuum results in recovery of up to 40% more oil as compared to facilities using vacuum pumps. Features of the compressor include an inlet about 0.1 bar (vacuum about 0.9 bar); outlet about 1.1 bar; capacity about 4 m³. The compressor is a screw-type compressor that does not use oil as a lubricant.

The volatile ether oil is extracted in the steam passing through a column in a distillation unit 20, i.e., through the fragmented herb/plant material in the buckets 22, and is carried to a drum shaped condenser 26 for condensation, which takes place in two stages. In the first stage, on leaving the distillation unit 20, the vapor enters the drum shaped condenser 26 where a majority of the oil is condensed. The condenser 26 is of a design that contains a receiver area that the vapor from the distillation unit first enters, and which ensures that the vapor is evenly distributed in all pipes. It should be noted that the condenser 26 is designed such that there are no sudden cross sections, large curves, or pockets where oil would be retained. Also, the condenser 26 has incorporated additional outlets or drains within the receiver to increase oil recovery. The outlets are arranged so that oil leaving the receiver contains a low water percentage, utilizing the differences in density between oil and water. Thus, the percentage of oil that is lost or retained in the receiver and condenser body is less than 0.1% of the total oil recovered from a single cycle of operation.

In the second stage of condensation, the remaining part of uncondensed water vapor and essential oil vapor, together with essential oil and water, enters a “tube to tube” second condenser/cooler 28 in which the condensation is completed. It then passes into glass decanters 30 (vessels for the separation of liquid). The decanters 30 are made of glass and allow the quantity of the ether oil to be measured. The advantage in having the tandem condensers 26 and 28 is their construction and proximity to each other that provides efficient transport of higher quality and increased amounts of the oil to the decanters. The oil will be transported from the decanters into stainless steel tanks 34 for immediate fractionation. The oil in the tanks 34 is moved into the fractionation column by forklifts.

The volatile ether oils are fractionated on large columns 32. Each column 32 is larger than previous columns and has bell-like bottoms on which the ether oil fraction will be separated. The increase in column size increases the quality and purity of the oil produced. The column 32 floors are oversized to decelerate the fractionation process and provide as great an exchange volume as possible between the liquid and gaseous phases/stages. An advantage of the present columns 32 is that the floors can be removed from the column itself to allow different oils, such as carvacrol, thymol and menthol to be purified. Each column 32 has outlets through which fractions will be transported to stainless steel tanks 34. The oil is transported into the large 34 tanks by pumps. These large tanks 34 are used for oil storage and transport within short time periods. The small tanks 36 are used exclusively for storage and include thermometers and are lined with insulation. They also have an extra opening in addition to the main one to allow samples to be taken for chemical analysis.

The quantitative content of carvacrol, thymol, and menthol can then be determined using chromatographic methods, such as, for example, solid-liquid, liquid-liquid, and gas-liquid type chromatography. Examples of solid-liquid type chromatographic methods that could be utilized include column chromatography, gel chromatography, dry-column chromatography, or high performance liquid chromatography (HPLC).

The present method of purification likely enhances the antimicrobial properties of the isolated carvacrol, thymol and menthol because of increased purity and quality. The coupled distillation and fractionation process increases the speed of purification and ensures the purity and quality of the final products, while reducing the cost of production of the final products.

III. Synthetic Production of Organic Phenolic Compounds and Essential oil Alcohols

Methods for synthetically producing organic phenolic compounds, such as carvacrol and thymol are well known in the art. Synthetic menthol can be prepared by the hydrogenation of thymol. Additionally, these compounds are available from chemical manufacturers and are listed in the Merck Index. Both synthetic carvacrol, synthetic thymol, as well as other synthetic organic phenolic compounds, are suitable for combining with either natural or synthetic menthol, to form antimicrobial compounds of the present invention. Additionally, synthetic menthol may be combined with other organic phenolic compounds, such as, for example, hypericine, natural tannins, and natural polyphenols. However, it is generally preferred that the organic phenolic compound, or essential oil alcohol, used in the present invention be extracted from plants instead of being chemically synthesized. Because phenol is used to synthesize carvacrol and thymol, the resulting synthetic products tend to contain residual phenol (less than 1%). It is undesirable to administer a phenol containing composition to an animal because phenol can be mutagenic and carcinogenic. Moreover, residual phenol may cause irritation, pain, discomfort, and damage in animals or humans.

IV. Antimicrobial Compounds

As used herein, the term “antimicrobial compound” refers to both unreacted organic phenolic compounds and compounds formed by reacting an organic phenolic compound extracted from a plant of the Lamiaceae, Labiatae and/or Verbenacae family with an essential oil alcohol, such as menthol. The natural compound menthol is an essential oil alcohol that can be purified from the volatile essential oil extracts of members of the Mentha genus, preferable Mentha piperita. In some instances, the antimicrobial compound formed by reacting an organic phenolic compound with menthol may be referred to as a “mentho reacted” compound. The antimicrobial compound may also be referred to as the “active ingredient or agent.” An antimicrobial compound may refer to a compound formed by chemically reacting carvacrol or thymol with menthol as illustrated in FIG. 1. As used herein the term “antimicrobial compound” also refers to unreacted carvacrol, thymol, menthol, hypericine, natural tannins, natural polyphenols, and combinations thereof. The term may also refer to the reduced forms of carvacrol, thymol, hypericine, natural tannins, and natural polyphenis. An “antimicrobial compound” may also refer the essential oil alcohol hexahydrothymol (menthol) or hexahydrocarvacrol (menthocarvol). In addition as used herein, the term “antimicrobial compound” also refers to the reduced forms of hypericine, natural tannins, natural polyphenols, and combinations thereof. It can also refer to combinations and mixtures of the above mentioned compounds.

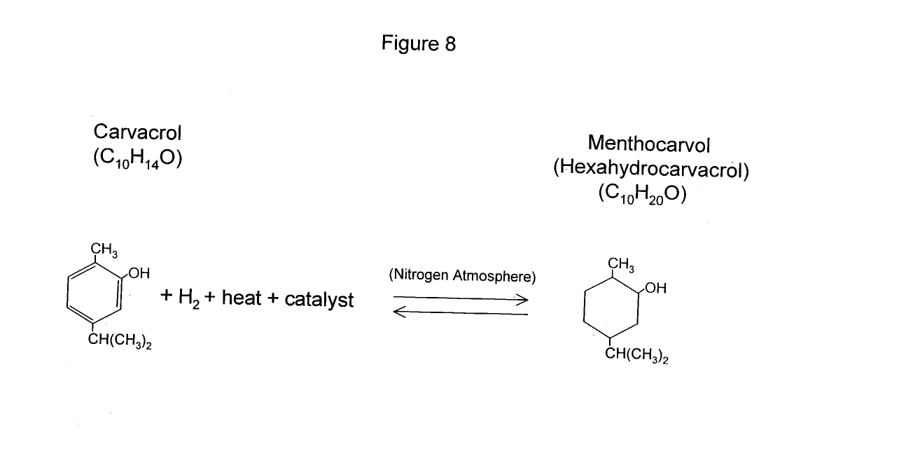

As used herein, the term “combining” refers to the process whereby compounds are brought into a close relationship to result in either a new chemical compound or a mixture in which all relevant properties of each individual compound are present. The term “reacting” as used herein, refers to a process in which an organic phenolic compound is chemically modified (as compared to the formation of a solution). In the formation of the new antimicrobial compound by reaction with a natural pharmacological agent menthol, a new chemical compound is made that is distinct and is believed to have increased antimicrobial activity and effectiveness (FIG. 1). In one preferred embodiment the resulting compound is called carvomenthol. In another embodiment the resulting compound is called thymomenthol. In still another preferred embodiment, the hexahydrogenated form of carvacrol is referred to herein as menthocarvol and is a novel therapeutic antimicrobial compound (FIG. 8). Mixtures of antimicrobial compounds may also be made to generate therapeutic pharmaceutical compositions of the present invention.

The “essential oils” of plants of the Lamiaceae, Labiatae and Verbenacae families also contain organic acids, alcohols, aldehydes, ketones, esters, other phenols, phenol esters, and more complex organic compounds that possess antimicrobial activities or other pharmacological activities. Examples of such organic acids include, but are not limited to, acetic benzoate, cinnamic, and phenylacetic. They include, but are not limited to, the alcohols benzyl alcohol, borneol, cinnamyl alcohol, citronellol, geraniol, linalool, phenylethyl alcohol, and terpineol. They include, but are not limited to the aldehydes, anisaldehyde, cinnamaldehyde, benzaldehyde, citral, piperonal, heliotropin, salicylaldehyde, and vanillin. They include, but are not limited to the ketones, carvone, camphor, menthone, thujone, and pulejone. Examlples of such organic acids include, but are not limited to the esters bornyl acetate, methyl salicylate, benzyl benzoate, geranyl acetate, and linalyl acetate. They include, but are not limited to the phenol, chavicol. They also include, but are not limited to phenol esters, anethol, eugenol, and safrol, as well as other more complex compounds. Each of these compounds may be combined or reacted with carvacrol, thymol, hexahydrothymol, hexahydrocarvacrol, or other like compounds to produce novel antimicrobial therapeutic compounds.

V. Reaction of Carvacrol and/or Thymol with Menthol

Carvacrol and thymol belong to a group of monoturpene phenolic compounds. It is well known that phenol, as well as alcohols, contain an OH group which is easily ionized, i.e., it easily releases the H+ (proton), which makes phenol group containing molecules weak acids. This acidic feature of the phenol moiety may be balanced by reacting the organic phenolic carvacrol and/or thymol with an agent, such as menthol to form a new compound via a nucleophilic addition reaction to form an aromatic ether compound. The resulting novel compounds, referred to herein, as carvomenthol, or as thymomenthol, are believed to enhance the antimicrobial activities of each in a synergistic manner, and additionally add the anesthetic and antispasmolytic activities of menthol to the compound. As shown in FIG. 1, carvacrol or thymol may be reacted and compounded with menthol. Other compounds that can be reacted with menthol to form a therapeutic composition of the present invention include hypericine and natural tannins or natural polyphenols. Hypericine is the active antimicrobial compound found in extracts from the plant Hypericum perforatum, commonly known as Saint John's wort, while a mixture of natural tannins is the antimicrobial compound found in the plant Salvia Officinalis, commonly known as English Sage. Natural tannins are also found in a wide variety of related plant species. Hypericine, natural tannins, or natural polyphenols may be reacted with menthol in reactions similar to those shown in FIG. 1.

Combining carvacrol and/or thymol with the menthol is aided by binding the two molecules into a complex to form an aromatic ether. Combining carvacrol or thymol with menthol increases the antimicrobial effect of the resulting compound because of the intrinsic pharmacological activity of menthol. Moreover the formulations act to reduce the irritation, discomfort, or gastrointestinal distress, associated with phenol-containing compositions, because of the mild local anesthetic and antispasmolytic activities of the menthol. The resulting antimicrobial compounds are thus more effective in praxis.

The preferred formulation reaction of carvacrol or thymol with menthol is believed to be equimolar, although other ratios are possible and may be tested empirically to find the most effective antimicrobial activity, while concomitantly reducing any potential side effects such as, irritation, discomfort, or gastrointestinal distress. The formulation reaction comprises thorough mixing of the two reactants by stirring in an appropriate vessel for about 30 minutes to 1 hour, or preferably 1 to 3 hours, or more preferably 3 to 6 hours, or for longer time periods, heating as needed. The addition of excess aqueous sodium hydroxide and the catalyst tetrabutylammonium hydrogen sulfate may be used to increase the efficiency of the reaction. Care should be maintained to prevent thermal degradation of the carvacrol, thymol, and menthol at high temperatures. The temperature is typically maintained between about 25-70° C. The reaction may also be conducted under a nitrogen atmosphere to minimize oxidation. If nitrogen is used, the temperature should be maintained as necessary, up to 180° C. The reaction may also use other catalysts such as, for example, quaternary ammonium salts, crown ethers, polyethylene glycols, or reaction conditions known in the art to facilitate the synthesis of the novel antimicrobial aromatic ether compounds of the present invention. These may include different phase transfer catalyzed condensation chemistry to make the aromatic ether compounds.

The antimicrobial compounds of the present invention should not have any significant toxicity. In one embodiment, the antimicrobial compound synthesized by the reaction of the pharmacological agent menthol with carvacrol (carvomenthol) is combined with an antimicrobial compound formed by reacting the same agent with thymol (thymomenthol). In some embodiments mixing may be required for 10 to 30 minutes, or from 30 minutes to 1 hour, or for longer periods of time, to ensure a complete and even composition. In a preferred formulation, the carvomenthol reacted compound is combined with the thymomenthol reacted compound in a ratio of approximately 90:10. In other embodiments, the carvomenthol reacted compound is combined with the thymomenthol reacted compound in other ratios, such as, for example of: 95:5, 85:15, 80:20, 75:25, 70:30, 65:35, 60:40, 55:45, 50:50 of carvomenthol to thymomenthol in the mixture. In still other embodiments, the individual compound percentages in the ratios of the two components can be reversed from the aforementioned ratios. Additional embodiments can contain only the carvomenthol reacted compound or thymomenthol reacted compound. Further embodiments can include hypericine reacted with menthol (menthohyperinice) and/or tannins reacted with menthol (menthotannin), and/or other polyphenols reacted with menthol (menthopolyphenol), in similar ratios shown above with either carvomenthol, thymomenthol, or combinations thereof. The different embodiments of the invention may also be combined with each other, as well as with the original carvacrol, thymol, and menthol, in various ratios to maximize therapeutic activity.

VI. Production of Menthocarvol (Hexahydrocarvacrol)

In addition to producing carvomenthol or thymomenthol from the reaction of carvacrol and menthol, it is possible to make hexahydrocarvacrol, referred to herein as menthocarvol, by the introduction of hydrogen atoms into the double bonds of the phenolic ring of carvacrol using a nitrogen atmosphere, hydrogen, a catalyst, and heat (FIG. 8). This reaction produces a novel antimicrobial compound that is distinct from carvacrol or carvomenthol but similar to menthol in structure. It is believed the menthocarvol has both enhanced antimicrobial activity while maintaining the anesthetic and antispasomolytic properties of menthol.

An experimental production of menthocarvol was performed from carvacrol isolated from purified oregano oil to evaluate the feasibility of this production method and the efficacy of this antimicrobial compound (oregano oil, Sigma-Aldrich Inc.). The starting sample of carvacrol was 98.7% pure. The carvacrol contained small amounts of impurities of thymol, α-pinene, p-cymol, thyme camphor, as well as a range of aliphatic compounds which are present in the natural oil and some of which function as preservatives.

Menthocarvol was produced in a glass reactor device with ground connections that allowed the introduction of gaseous nitrogen into a standard cylinder vessel using bi-phase regulation of the outlet pressure. The cylinder chamber allowed work to proceed in a nitrogen atmosphere with a heating system capable of reaching 300° C. The nitrogen atmosphere was used because of the volatility of the carvacrol, which is easily oxidized under normal air atmospheric conditions.

An initial analysis was done to set parameters for the production of menthocarvol form carvacrol. Different, catalysts, i.e., nickel, Raney-nickel, and platinum, were evaluated in preliminary experiments. Based on evaluations of the proposed reaction mechanism estimates of yield for these catalysts were 41%, 63%, and between 84-88%, respectively. In one embodiment of the reaction 142 g of pulverized platinum, with a particle size ranging from 2-4 microns, was used for the reaction of 10 g of carvacrol. The temperature of the reaction was set to be between about 160° C. and 180° C. based on the stability of carvacrol at elevated temperatures. The hydrogen pressure of the reaction was adjusted to be between about 2.5-4.5 bars. The time of reaction was preferably between about 4-6 hours (h).

The reaction was initiated by transferring the carvacrol sample into the reactor vessel having a nitrogen atmosphere and catalyst. The reaction mixture was heated and stirred throughout. The reaction was allowed to reach 170° C. at which point hydrogen was allowed to flow into the reaction vessel. The hydrogen flow was maintained at a pressure of approximately 3.2 bar. The reaction was maintained under these conditions for 6 h after which the reactor mixture was cooled to about 25° C. In one embodiment, the catalyst platinum was precipitated and the reactor content was transferred to a filtration device. The solid phase was separated from the oil fluid phase, and the oily fluid analyzed by gas mass chromatography (GMC, HP-GL-1900 CGCD) using an apparatus with standard settings.

The analysis confirmed that menthocarvol was produced with a yield of approximately 85% based on content. All mixture fragments were not separated. Analysis revealed that the reaction mixture contained approximately 85.0% menthocarvol, 12.5% carvacrol, and 2.5% of α-pinene. In addition p-cymol, and thyme camphor traces were detected. It is estimated that some undetected partially reduced carvacrol molecules are also present in the reaction mixture.

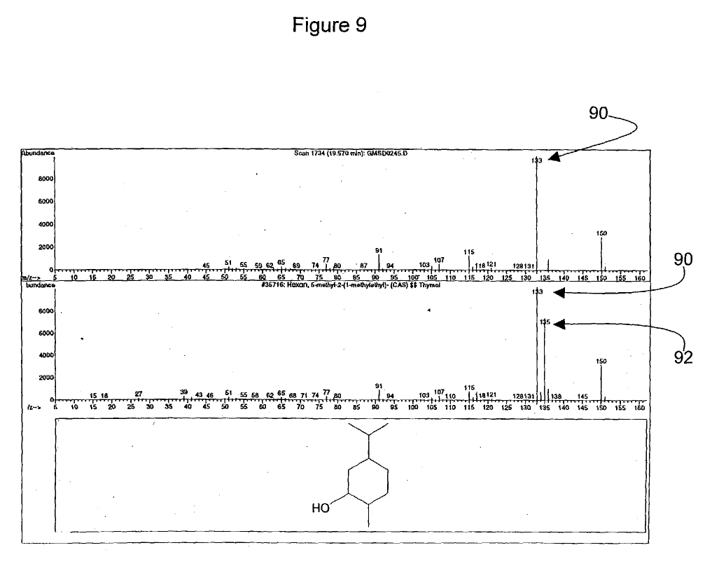

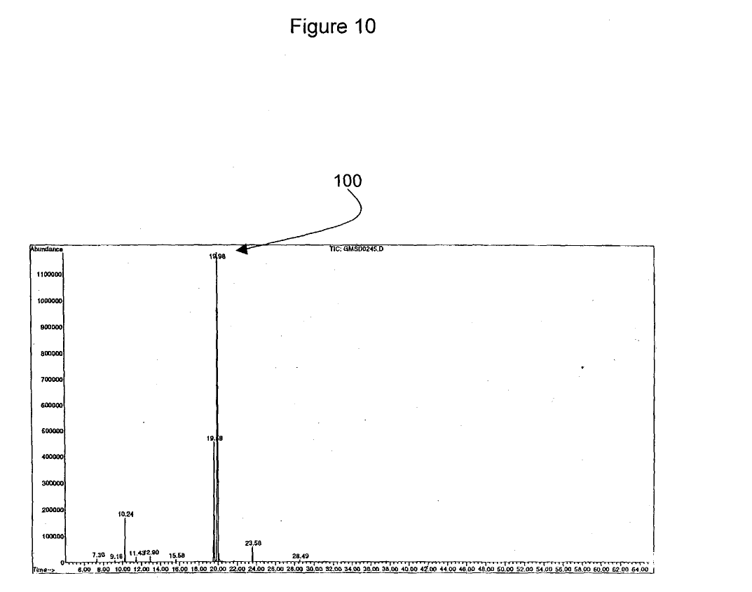

The major product menthocarvol represents a compound that is of an oily consistency with significant changes with respect to odor compared to carvacrol and menthol. The graphical representation of a comparative analysis is shown in FIG. 9, wherein, menthocarvol is indicated by gradient 90 (133) and the starting material carvacrol is indicated by gradient 92 (135). The peak of the new compound menthocarvol is shown in FIG. 10 and is indicated by gradient 100 (19.98). Other peaks present in the chromatograph in FIG. 10 are residues of carvacrol reacted with impurities in the original starting material. The compound menthocarvol has unique physical properties compared to carvacrol.

Menthocarvol

| | |
|---|---|
| Molecular weight | 156.27 |
| Specific rotation | a + 31.4° |
| Boiling point | 222.102° C. |
| Density | ρ = 0.8995 |
| Refraction index | η = 1.4617° |

Soluble in diethyl ether or ethanol.

VII. Pharmaceutical Compositions, Therapeutics, Medicaments, and Medicinal Agents

The antimicrobial compounds of the present invention can be used alone, or as part of a pharmaceutical composition, therapeutic, medicament, or medicinal agent. These terms are defined as interchangeable, and as used herein, these terms refer to a composition that includes at least one antimicrobial compound and a pharmaceutically acceptable carrier. The term “carrier” refers to the overall inert components in the formulation of a therapeutic and may include the following: diluents, binders, lubricants, disintegrants, emulsifiers, coloring agents, and flavoring agents, which together in a final form optimize delivery of the therapeutic antimicrobial to the desired body location and site of infection. Some of these classes of compounds are overlapping and individual compounds may possess one or more of these activities in different degrees of activity. They are typically used in combinations with an active ingredient (i.e., the antimicrobial therapeutic compounds of the invention) and their formulation into pharmaceutical compositions are well known in the art. The term “pharmaceutical composition, therapeutic, medicament, or medicinal agent” can refer to a combination of an unmodified organic phenolic compound and/or menthol reacted organic phenolic compound and a pharmaceutically acceptable carrier. This definition of “pharmaceutical composition, therapeutic, medicament, or medicinal agent” includes essential oils obtained from plants as well as synthetically produced organic phenolic compounds combined with acceptable carriers. It also refers to the menthol reacted compounds carvomenthol, thymomenthol, menthohypericine, menthotannin, and menthopolyphenol, among other similar reacted compounds, combined with acceptable carriers. It also includes menthol and/or menthocarvol and/or other essential oil alcohols, combined with an acceptable carrier. It also includes embodiments where menthol may be substituted for by molecules with similar pharmacological properties in the compounds listed above, together with an acceptable carrier.

The therapeutic pharmaceutical compositions of the present invention may be administered orally and topically to a subject. In oral preparations, absorption of the antimicrobial compound is primarily through the gastrointestinal tract. Oral preparations may be formulated capsules, tablets, liquid syrups, mouth washes, or gums, troches using methods that are known in the art. Topical preparations of the present invention are absorbed primarily through the skin. Topical preparations may be formulated as eye drops, ear drops, nasal inhalants, powders, creams, lotions, ointments, shampoos, and sprays using methods that are known in the art.

Typically, the preparation of a parenteral formulation of a pharmaceutical composition, therapeutic, medicament, or medicinal agent begins with the selection of the carriers to be used. If in powdered forms a carrier can be finely ground and mixed evenly with a similarly finely ground and divided active ingredient. If in tablet form, the carrier and active ingredient are formed to have the necessary properties relating to binding, form and hardness for formation into unit dosages. The carrier typically has no therapeutic activity. Absorption of the therapeutic from the carrier can be affected by the viscosity of the carrier, its capacity for wetting the solid particles, the solubility equilibrium produced by the carrier, and the distribution coefficient between the carrier and the aqueous system of the body.

Medicinal agents are often taken orally in a form of a tablet or capsule. Their base formulation, methods of manufacture, and commercially available equipment used for such, are well known in the art. Tablets are solid pharmaceutical dosage forms prepared by molding or compressing methods. They may be composed of different shapes and hardness whose overall composition affects the availability of the therapeutic agent. Common types include, compressed tablets, sugar-coated, film-coated, enteric-coated, multiple-compressed tablets, controlled release tablets, triturates, among others. Tablets often contain a variety of ingredients that are inert therapeutically that affect the physical characteristics of the formulation. These include diluents, binders, glidants, and lubricants. They also often include coloring and flavoring agents.

Diluents increase the bulk of the tablet and include, but are not limited to, dicalcium phosphate, calcium sulfate, lactose, cellulose, microcrystallin cellulose, cellulose derivatives, kaolin, mannitol, sodium chloride, corn starch, other dry starches, glucose, sucrose, and other powdered sugars. In most cases of immediate release tablets only one or two of the diluents are used, but in some forms consideration of the interaction of the medicinal agent and these compounds needs to be contemplated. In time or controlled release forms the polymer hydroxypropylmethylcellulose may be used, as well as other like polymers. This confers prolonged release characteristics and is also added as a film former in the tablet coating.

Binding agents add physical cohesive properties to the tablet. They are also referred to as granulators. Commonly used binders include, but are not limited to, natural/synthetic gums including acacia, sodium alginate, Irish moss extract, panwar gum, ghatti gum, mucilage of isapol husks, gelatin, carboxymethylcellulose, methylcellulose, other cellulose derivatives, polyvinylpyrrolidone, veegum, and larch arabogalactan. Other compounds sometimes used as binders include polyethylene glycol, waxes, alcohols, and water. Many of these are polymeric in nature. The component binders affect the hardness and disintegration properties of the compressed tablet. They can be used as dry powders or in solutions depending on other ingredients of the medicament tablet. Typically, binders are used in weight to volume ratios. Corn starch is commonly used at between about 10-20% as a paste in water and has the added properties of including some disintegrate material into the medicinal granule. Gelatin solutions are often used at about a 10-20% solution and are used while warm or they typically will solidify. Cellulose derivatives are often employed and include hydroxypropylmethycellulose, hydroxyethyl cellulose, and hydroxypropylcellulose. These are dissolved in water or sometimes alcohols, as in the case of ethylcellulose, and are typically used with materials that are moisture sensitive. Polyvinylpyrrolidone can be used in aqueous or with alcoholic solutions and its useful concentrations vary from about 2-20%.

Lubricants serve several functions in medicinal tablets. One is that they prevent sticking of material to equipment used in manufacturing, dies and the like. Lubricants are divided finely by passing through mesh nylon cloth onto the granulation. The time and order of addition of specific lubricants can affect the overall tablet's physical properties. Typical lubricants used include, but are not limited to, talc, magnesium stearate, calcium stearate, stearic acid, hydrogenated vegetable oils, and polyethylene glycol (PEG). Most lubricants are used at about 1% or lower concentrations, except talc that is used at levels up to about 5%. Care must be given to selecting the ideal lubricants and should consider the entire scope of components used in the formulation.

Disintegrants are added to facilitate the breakup of the tablet. They are individual materials or mixtures in these substances. These compounds are believed to work by their swelling characteristics among other properties. Corn, potato, or other starch source, is a commonly used such agent and is used from about 5-20% in a powdered and well dried form. Such starches often serve dual roles as binders and distintigrants. Other extremely effective disintegrants include crosslinked polymers, croscarmelose, crospovidone, and sodium starch glycolate. In addition, still other materials can add disintegrating activity and include Veegum HV, methylcellulose, agar, bentonite, natural sponge, alginic acids, guar gum, citrus pulp, among other similar materials. Such agents are typically mixed with the active ingredients and diluents prior to granulation. The choice and method of combining disintegration agents often depends on the complete medicinal formulation.

Emulsifiers are often used in medicaments. They reduce surface tension, increase absorption, increase viscosity, and are effective at low concentrations. One important characteristic is the ability to form a film around each droplet of dispersed material. Synthetic, natural, and finely divided solid emulsifying agents may be employed. Synthetic emulsifying agents include, but are not limited to, potassium laurate, triethanolamine sterate, sodium lauryl sulfate, alkyl polyoxethylene sulfates, and dioctyl sodium sulfosuccinate. Naturally derived agents include, but are not limited to, acacia, gelatin, and lecithin. The finely divided solid emulsifiers include, but are not lmited to, colloidal clays, bentonite, veegum, magnesium hydroxide, other metallic hydroxides, and silicone dioxide. Some compounds are considered auxiliary emulsifying agents because they aid other agents, but are not entirely capable themselves. They include, but are not limited to, cetyl alcohol, glyceryl monosterate, methylcellulose, sodium carboxymethylcellulose, other cellulose derivatives, and steric acid. Sometimes emulsifying agents are referred to as stabilizers, suspending agents, or thickening agents. Many are used where emulsification of ingredients is desirable, and are common in topical ointments of various types.

Coloring agents typically include the approved water soluble FD&C dyes and their mixtures. The main purpose of their use is to make the tablets more pleasing to the eye and to establish identity. Other approved colorants are well known in the art. Flavoring agents are often used in conjunction with coloring agents and add pleasing taste to tablets and often include mannitol, lactose, glucose, sucrose, and artificial sweeteners.

Several manufacturing methods for making tablets are popular and known in the art. These include the wet, dry, and fluid-bed granulation processes. Other related granulation methods include spheronization, spray-drying, and spray-congealing, which employ techniques that aid in the formation of a homogenous and uniform granulation.

The wet granulation process typically consists of steps of weighing and mixing the dry ingredients including the active ingredient, diluent, and disintegrating agent. These may be sifted through a screen to ensure a fine and even mixture. This is followed by the granulation process where solutions of the binding and other carrier agents are added to the mixed powders. The wet granulation is typically pushed through a mesh screen or sieve. Commercial mills which may include rotary devices, or mixing equipment, which use blades, hammers, knives, or oscillating bars, often are employed in large scale preparations. The granulation is then dried on trays or with fluid-bed driers. Some degree of moisture is maintained in the granulation during the drying process. The particle size is usually reduced by passing through screens after drying and combined with a lubricant and then compressed into tablets.

In the related fluid-bed granulation method, the granulating solution is sprayed onto suspended particles of the active ingredient, which are suspended in a column of air. This provides a rapid process to form granules of a desired size that are dried during the process and that are ready for compaction with a specified lubricant. The exact parameters of this process will vary with equipment and formulation of the medicament.

Dry granulating methods are used when the ingredients are sensitive to moisture or heat used during the drying process in other granulating methods. The active ingredient, diluent, and part of the lubricant are mixed. One of these components usually has cohesive properties. Air is expelled under pressure and a dense material is formed. This is often referred to as a slug or slugging. Direct compression is similar to dry granulation, except the dry powdered ingredients are not modified and so the method is limited to compounds with desirable physical properties relating to cohesion and flow.

Capsules are solid forms of therapeutics that are encased in either a hard or soft shell container. These are usually tasteless and easily filled by machinery and methods known in the art. The dosage of capsules is easily adjusted to individual prescription needs. The shells are readily commercially available in a variety of convenient sizes and are made out of gelatin, ethylcellulose, or like materials. The soft gelatin shell is often thicker than the hard one and employs a plasticizer that is usually glycerin, sorbitol, or similar polyol. It is important that the medicament be a uniform dry mixture which is usually done by trituration, reducing it to a fine powder. In eutetic mixtures with low melting points, it is typical to include a suitable absorbent agent such as magnesium carbonate.

Microencapsulation is another method that allows the coating of particles of the antimicrobial active ingredient with thin coatings to produce solids, droplets of liquids, or dispersions. Several materials can be used in this process and include gelatin, polyvinyl alcohol, ethylcellulose, cellulose acetate pthalate, other cellulose derivatives, styrene maleic anhydride, among other similar materials. The microcapsule may be one or a combination of several particles or a cluster: After manufacture the material appears as a powder with excellent flow characteristics. It is commonly used to mask undesirable taste properties and to form prolonged acting release forms as it is known in the art.

Liquid form preparations include solutions, suspensions, and emulsions, for example, water or water-propylene glycol solutions for oral or topical administration. Such solutions are prepared so as to be acceptable to biological systems (isotonicity, pH, hydrophobicity, viscosity, among other solution properties). Liquid preparations can also be formulated in aqueous polyethylene glycol solutions. Aqueous solutions suitable for oral use can be prepared by dissolving the antimicrobial compound in water and adding suitable colorants, flavors, stabilizing, emulsifying, and thickening agents as desired. Aqueous suspensions suitable for oral use can be made by dispersing finely divided active components in water with said viscous material, such as natural or synthetic gums, resins, methyl cellulose, carboxymethyl cellulose, as well as other accepted cellulose derivatives, and other known suspending agents and like compounds. Commonly used medicinal aqueous solutions include, but are not limited to, syrups, elixirs, gargles, mouthwashes, nasal solutions, otic solutions, ophthalmic solutions, irrigation solutions, and inhalants, all of whose formulation and manufacture are well known in the art.

Topical ointment preparations may contain formulations of the antimicrobial compound for topical uses to treat infections. Four broad types of ointments, referred to as ointment bases, are used to deliver medicinal agents. The first is hydrocarbon base and usually includes petrolatum, which is a unctuous material having emollient qualities and a melting temperature of about 38-60° C. They may also include oleaginous base along with bases made from vegetable oils or animal fats. These ointments often require addition of antioxidants and other preservatives are not as commonly used today.

A second ointment type is referred to as absorption bases. These are hydrophilic and may include anhydrous materials or alternatively hydrous bases that can absorb water. These often include lanolin, which is a complex assortment of compounds that readily absorb water. Other components of absorption bases may include stearyl alcohol, white wax, and white- petrolatum. Derivatives of lanolin are available and include alcohols, dewaxed, acetylated, ethoxylated, hydrogenated, among others types of modifications. In some absorption bases cholesterol has been substituted for lanolin due to allergic reactions reported to lanolin type compounds. There are several commercially available absorption bases including, but not limited to, Aquaphor, (Beirsdorf), Polysorb (Fougera), and Nivea (Beirsdorf).

A third ointment type, commonly referred to as creams, are water removable base or emulsion bases. These typically have three main components: an oil phase, the emulsifier, and the aqueous phase. The medicinal agent may be part of any of the three phases or alternatively added to the final emulsion. The emulsion base hydrophilic ointment USP is an example designed to minimize any undesirable drug interactions, either physical or chemical. It contains methylparaben, propylparaaben, sodium laurel sulfate, propylene glycol, stearyl alcohol, white petrolatum, and water. It is typically made according to established formulas that are well known in the art. In this illustrative example the sterayl alcohol and petrolatum make up the oil phase and give the desired smoothness and characteristics for compatibility with the skin. An enormous number of additional oil phase components are known in the art and may be used. Preservatives may be added to the aqueous phase and include, but are not limited to, methylparaben, propylparaben, benzyl alcohol, sorbic acid, or quaternary ammonium compounds. The aqueous phase in most cases exceeds the oil phase and contains the preservative materials and emulsifiers. The emulsifier may be either an anionic, cationic, or nonionic type. The aqueous component also contains the humectant, which is often glycerin, propylene glycol, or a polyethylene glycol. These add to the overall characteristic of the preparation and maintain water content. Any necessary components for maintaining pH, buffers, antioxidants, and stabilizers are also included in the aqueous phase.

The fourth type are the soluble ointment bases that are composed of soluble ingredients. These may also be gelled aqueous solutions, which are commonly called gels. The polyethylene glycols are often the major component of these water soluble bases and are known to be inert and specifically do not cause irritation. These have characteristics ranging from liquids to soft solids to hard wax types. Commonly used polyethylene glycols include polymers designated by their average molecular weights of 400, 1500, 1600, and 6000. Various gelling agents are often included and include: cellulose derivatives, carbomers, colloidal magnesium aluminum silicate (Veegum), sodium alginate, and the propylene glycol ester of alginic acid (kelcoloid). One feature of the soluble ointment bases is that they are designed to maximize delivery of the therapeutic agent.

The therapeutic composition may also contain additives such as preservatives, vitamins, minerals, and amino acids. Suitable additives may vary depending on the desired end use for the pharmaceutical composition. They may also contain fats or oils that have nutritional or metabolic value, that include ω-3-fatty acids, among other like compounds, believed to lower plasma low-density lipoprotein and cholesterol levels. Examples include olive oil, fish oil, soybean oil, and rapeseed oil. Vitamins that may be included in the pharmaceutical composition include vitamins of the A group (Retinol, Retinoic acid, Dehydroretinol, and the Provitamin A carotenoids), B group (Thiamin, Riboflavin, niacin, Pantothenic acid, Biotin, Folacin, and B₁₂), C group (_{L}-Ascorbic acid, and Antiscorbutic vitamin, D group (Antirachitic vitamin, D₂, and D₃), E group (occur as fatty acid esters ), and K group (K₁, K₂, K₃, K₄₋₇). Nutritionally important elements including important trace ones may be added to the therapeutic composition and include: Calcium (Ca), Sodium (Na), Potassium (K), Cobalt (Co), Copper (Cu), Chromium (Cr), fluorine (F), Iodine, (I), Iron (Fe), Magnesium (Mg), Manganese (Mn), Molybdenum (Mo), Nickel (Ni), Selenium (Se), Silicon (Si), Tin (Sn), Vanadium (V), and Zinc (Zn). Additionally, any of the naturally occurring 20 amino acids may be included in the therapeutic composition.

The typical therapeutic or pharmaceutical composition will be in unit dosage form. In such cases, the preparation is subdivided into unit doses containing appropriate quantities of the antimicrobial compound. The unit dosage form can be a packaged preparation, the package containing discrete quantities of preparation, for example, packaged tablets or capsules, in specific weight amounts. Tablets, capsules, cachets, troches, therapeutic powders, ointments, creams, syrups, elixirs, gargles, mouthwashes, nasal solutions, otic solutions, ophthalmic solutions, irrigation solutions, inhalants, and other medicinal liquid preparations can typically contain from about 1 wt % to about 45 wt %, or preferably about 2 wt % to 20 wt %, or more preferably about 3 wt % to 15 wt %, and most preferably about 4 wt % to 10 wt % antimicrobial compound.

The quantity of antimicrobial compound in a unit dose may be varied or adjusted from 1 mg to 1000 mg according to the particular application. The antimicrobial compounds are typically administered at an initial dosage of about 5 mg to about 150 mg per kilogram daily. The dosages, however, may be varied depending upon the requirements of the individual being treated, the severity of the condition being treated, and the compound employed, as well as the medicinal delivery mode used. Determination of the proper dosage for a particular situation is within the skill of the art. In most cases treatment is initiated with smaller dosages based on the individual, which can be increased by small increments until the desired therapeutic effect is obtained. For convenience, the total daily dosage may be divided and administered in portions during the day if desired.

Particular tablet, capsule, or microcapsule layered formulations that allow delayed release of the antimicrobial therapeutic are desirable. These would ideally allow the antimicrobial agent access to the large intestine. Products based on essential oils, such as organic phenolic compounds, tend to be absorbed at a level greater than 90% in the small intestines. Therefore, most of the activity of such products tends to be localized in the stomach and/or small intestine. Nevertheless, there are many microbial infections that occupy portions of the gastrointestinal tract beyond the small intestine. Therefore, it may be desirable to extend the activity of therapeutic agent into the large intestine. These embodiments could be in the form of prolonged release tablets, capsules, including microcapsule layered forms, designed to release a majority of the antimicrobial agent in the desired region of the gastrointestinal tract. These could utilize the differences in pH in different areas of the gastrointestinal tract to provide the desired release of the medicament. For example, release could be optimized for differences in the pH along the gastrointestinal tract, which varies throughout: in the stomach, the pH is between 2 and 5; in the duodenum, 4 and 6; jejunum, 4 and 6; ileum, 6.5 and 7.5; caecum 5.5 and 6.5; colon, 6.5 and 7; and rectum, 6.5 and 7. Therefore, the components of the tablet, capsule, or microcapsule wall layers, may differ depending on what type of an ailment is to be treated, or its location. Each tablet, capsule, or microcapsule wall layer, also would contain the compound of the invention so that upon dissolution, it can be released to effect treatment of the ailment.

VII. Illustrative Therapeutic Pharmaceutical Compositions Containing Antimicrobial Compounds for Administration to Humans

Several different formulations of the antimicrobial compounds of the invention are possible. Presented below are some illustrative examples of formulations.

A. Oral and Topical Formulations

The antimicrobial compounds can be formulated into an ingestible oral preparation. For such preparations, the antimicrobial compound can be present at varying concentrations, for example, from about 1 wt % to about 45 wt %, or preferably about 2 wt % to 20 wt %, or more preferably about 3 wt % to 15 wt %, and most preferably about 4 wt % to 10 wt % antimicrobial compound. The antimicrobial compound can be utilized within these illustrative ranges with the carrier agents making up the remainder. Preferably, the carriers are nonaqueous. The antimicrobial compound and the carrier are typically combined in a commercial stainless steel mixer with two blades positioned at 90 degree angles to each other (TBI-27 Techno Industrie GmbH, Germany), or with similar equipment, stirred at approximately 500-2,500 revolutions/minute for about 5-30 minutes. The exact parameters of making specific formulations, including components, time, equipment, and carriers used in the manufacture of the antimicrobial compounds will vary depending on the formula and intended use of the particular embodiment. Equipment and methods for manufacturing therapeutic tablets, capsules, cachets, powders, ointments, creams, syrups, elixirs, gargles, mouthwashes, nasal solutions, otic solutions, irrigation solutions, and other medicinal liquid preparations are well known in the art and may include fluid bed granulizers, spray emulsion mixing techniques, as well as sophisticated heating, mixing, blending, and combining equipment, together with accepted pharmaceutical techniques for optimizing their manufacture. After the antimicrobial formulation is compounded, it is sterilized, preferably with ultraviolet radiation. After sterilization the therapeutic may be administered to a subject, or packaged for storage.

B. Illustrative Example Formulations

The following examples are illustrative formulations of different oral or topical embodiments of the antimicrobial therapeutics of the present invention. Other formulas for both liquid oral and topical ointments are contemplated and the methods, carrier ingredients, and method of manufacture, are well known to one skilled in the art.

1. Microencapsulation Formulation

Fluidized bed coating can be used to encapsulate the antimicrobial compound in a coating material, which includes ethyl cellulose and plant oil. In one embodiment, the antimicrobial compound is combined in the fluid bed mixer with the following ingredients to form a powder:

| Ingredient | weight % |
|---|---|
| Antimicrobial Compound | 10-15 |
| Corn Starch | 30-40 |
| Dextrose | 35-45 |
| Calcium Carbonate | 1-5 |
| Sodium Bicarbonate | 1-5 |
| Silicon Dioxide (SiO₂) | 1-5 |

The mixture is combined for about 10 to 30 minutes, preferably about 15 to 20 minutes at a temperature between about 75° C. and 100° C., more preferably between about 80° C. and 90° C. to form a powder.

After the powder is formed, a fatty acid composition can be added to the fluid bed mixer and mixed for about 5 minutes to about 20 minutes, more preferably about 10 minutes to about 15 minutes to form a fatty acid layer on the antimicrobial powder. Generally, the fatty acid is included in the mixer in an amount of about 20 wt % to about 25 wt % fatty acid composition as compared to about 75 wt % to about 80 wt % antimicrobial powder.

After the fatty acid layer is formed, ethylcellulose is added to the mixer and mixed for about 5 minutes to about 20 minutes, more preferably about 10 minutes to about 15 minutes, to form an ethylcellulose layer. Ethylcellulose is added to the mixer in an amount of about 20 wt % to about 25 wt % ethylcellulose and a suspension is formed by combining about 75 wt % to about 80 wt % coated antimicrobial powder.

As used herein, the term “fatty acid composition” refers to aliphatic monocarboxylic acids, and similar compounds, that can be liberated by hydrolysis from naturally occurring fats and oils, as well as synthetic fats and oils, including synthetic compounds that have modifications or are derivatives. Fatty acids are predominantly straight-chain acids of carbon atoms that may be saturated or unsaturated. The fatty acids are typically derived from plants, such as an oil seed, or animal sources, such as tallow, or are available as synthetic compounds from a variety of commercial sources. Examples of saturated fatty acids include, but are not limited to, palmitic and stearic acids, and their natural or synthetic derivatives. Examples of unsaturated fatty acids include, but are not limited to, oleic acid, and linolenic acid, and their modified derivatives. Examples of suitable plant oils include, but are not limited to, rapeseed oil, corn oil, peanut oil, safflower oil, olive oil, soybean oil, sunflower oil, cottonseed oil, menhaden oil, herring oil, peanut oil, crambe oil, mustard oil, meadowfarm oil and canola oils.

Additional layers are formed by adding alternating rounds of fatty acid and ethylcellulose to the mixer in the same manner as described previously. Preferably, the encapsulated antimicrobial compound is prepared with about 2 layers to about 10 layers, more preferably about 4 to about 6 layers. As used herein, “layer” refers to either a fatty acid, ethylcellulose, hydroxypropylmethylcellulose, or similar cellulose derivative, or similar polymeric compound layer. Multiple layers can be made up of these compounds, either alone or in various combinations and thicknesses, depending on the desired pharmacological parameters affecting release of the antimicrobial therapeutic. Generally, the resulting microcapulse has a diameter ranging from about 2 to about 5000 μm.

The microencapsulated product will allow a small liberation (about 20 wt % to about 30 wt %) of antimicrobial compound (active ingredient) in the stomach with the remainder being released in the small intestine, ileum and even in the colon depending on the specific compounds and number of layers used.

2. Capsule

To form a capsule for release of the antimicrobial compound in the intestine as opposed to than the stomach, the antimicrobial compound may be prepared first as a powder and encapsulated. To form a powder, the antimicrobial compound may be combined with the ingredients in the relative amounts shown below:

| Ingredient | weight % |
|---|---|
| Antimicrobial Compound | 10-15 |
| Corn Starch | 25-35 |
| Dextrose | 40-50 |
| Calcium Carbonate | 1-5 |
| Sodium Bicarbonate | 1-5 |
| Other* | 1 |
| Silicon Dioxide (SiO₂) | 1-5 |

(*Includes vitamins, minerals, amino acids and other additives)

The ingredients are preferably combined in the order shown. After each ingredient is added, the combination is mixed for 3 minutes at 500 rpm. To form the capsule, the encapsulated powder may be then combined with a binder, such as dextrose and potato starch in the amounts shown below:

| Ingredient | weight % |
|---|---|
| Encapsulated Powder | 10-15 |
| Dextrose | 35-45 |
| Potato Starch | 45-55 |

After the capsule is formed, it may be encapsulated with a coating material. An illustrative coating material includes gelatin or ethylcellulose and soy bean oil.

A 500 mg capsule (containing the ingredients in the ratio shown above) can be formulated and used to treat infections of the digestive tract in humans. An illustrative dosing regimen includes administering one 500 mg capsule daily to children (up to 12 years old) or two 500 mg capsules to adults every 8 hours.

3. Tablet

Tablets may be formulated by combining the encapsulated powder described above with dextrose, potato starch and colloids in the amounts shown below. Colloids are neutral ingredients which act as a glue to hold the active ingredients together.

| Ingredient | Weight % |
|---|---|
| Encapsulated powder | 10-15 |
| Dextrose | 35-45 |
| Potato Starch | 35-45 |
| Colloids | 10-15 |

The tablet may also include other carriers such as ethylcellulose or lactose. In one embodiment, a 500 mg tablet may be formulated and used to treat digestive tract ailments in humans by administering one tablet daily to children up to 12 years of age, and two tablets every 8 hours for adults, until symptoms, such as diarrhea, subside.

4. Slow Release Tablet

The tablet form of the antimicrobial compound may be prepared as a slow release formulation to treat, for example, urinary tract infections. In such embodiments the slow release formulation contains additional ethylcellulose (5% more than previous formulation) to enhance activity in the intestine.

The antimicrobial compound is prepared as an encapsulated powder and combined with carriers as shown below:

| Ingredient | Weight % |
|---|---|
| Encapsulated powder | 10-15 |
| Silicon Dioxide (SiO₂) | 10-15 |
| Potato Starch | 35-45 |
| Lactose | 15-25 |
| Dextrose | 15-25 |

One 500 mg tablet may be administered to children (ages 5-12 years) every 6 hours for 7 days. Adults may receive 3 tablets every 6-8 hours for 7 days, or for longer periods of time.

5. Syrups

Encapsulated powdered antimicrobial compound may be formed as described above and included in a syrup. An example of a syrup formulation is provided below:

| Ingredient | Weight % |
|---|---|
| Encapsulated powder | 10-15 |
| Tween 200 | 35-45 |
| Polysorbate | 25-35 |
| Honey Liquid | 15-20 |

Other carriers that can be included in the syrup include flavorings, such as vanilla flavor, strawberry flavor, honey flavor, orange flavor, among others. Preferably, the carriers are based on sugar products, such as fructose, dextrose, and other sugars. Generally, these carriers are added to cover any unpleasant taste (generally bitter) associated with the active ingredients. The syrup can then be used to treat digestive ailments, for example, by administering 2 ml of syrup containing 3-5 wt % antimicrobial compound orally every 8 hours, or for longer time periods.

6. Liquid Formulations

The antimicrobial compound can be formulated as a liquid for use in treating oral infections in humans by combining antimicrobial compound with carriers as shown below:

| Ingredient | Weight % |
|---|---|
| Antimicrobial Compound | 5-10 |
| Polysorbate | 15-25 |
| Propylene Glycol | 70-80 |

The liquid is administered to the area of the infection three times daily until the infection subsides.

7. Evaporated Solution

The antimicrobial compound can be formulated as an evaporated solution for treating tuberculosis in humans. To prepare this formulation, a liquid solution containing the antimicrobial compound is first prepared as described above. The liquid solution is then evaporated. The evaporated solution is then combined with the carriers shown below to form a composition that can be inhaled using a mask:

| Ingredient | Weight % |
|---|---|
| Antimicrobial compound | 15-20 |
| Ethyl alcohol | 15-20 |
| Propylene glycol | 25-35 |
| Double distilled Water | 35-45 |

Three to five ml of the formulation is then combined with 500 ml of hot water. The patient inhales the steam for 20 minutes, once a day for 10 to 15 days. The patient can repeat the treatment for 10 more days or longer if the tuberculosis persists.

8. Powder

The antimicrobial compound can be formulated as a powder suitable for treating athlete's foot and other microbial or fungal infections. To prepare a powder formulation, a liquid composition is prepared as described above and combined with carriers as shown below:

| Ingredient | Weight % |
|---|---|
| Antimicrobial compound | 5-10 |
| Potato Starch | 35-45 |
| CaCO₃ | 50-55 |

The powder can be liberally applied to the affected area three (3) times per day for 7 to 10 days.

9. Ointment Gel

The antimicrobial compound can be formulated as a gel for treatment of external infections caused by fungi and bacteria. To prepare a gel, a liquid formulation of the antimicrobial compound is first prepared as described above and then combined with carriers shown below:

| Ingredient | Weight % |
|---|---|
| Antimicrobial compound | 5-10 |
| Soya oil | 30-35 |
| Vaselinum album | 55-60 |

The gel is applied to the affected area three times a day for 5 to 7 days, or for longer time periods.

10. Shampoo

The antimicrobial compound can be formulated as a shampoo for the treatment of skin infections affecting the head and scalp. The shampoo can be formulated by preparing liquid formulation as described above and then combining the liquid formulation with ingredients to produce a shampoo as shown below:

| Ingredient | Weight % |
|---|---|
| Antimicrobial compound | 5-10 |
| Shampoo carrier | 40-50 |
| Polyethylene glycol | 45-55 |

The hair and affected scalp should be washed once a day for 10 days with the shampoo formulation. Alternatively, the hair and scalp can be treated with a liquid formulation prepared as described above and combined with corn oil as shown below.

| Ingredient | Weight % |
|---|---|
| Antimicrobial Compound | 5-10 |
| Corn Oil | 90-95 |

The liquid formulation can be applied via dropper, 5 drops to the affected area every 8 hours for 3 to 4 days.

IX. Infections

The therapeutic pharmaceutical compositions of the present invention can be used to treat a variety of internal and external infections in subjects, including humans and other animals. For example, the therapeutic pharmaceutical compositions of the present invention, may treat infections caused by, among others: Escherichia spp. including: E. coli, Salmonella spp., Shigella spp., Aeromonas spp., Pasteurella spp., Plesomonas spp., Staphyloccocus spp., Streptoccocus spp., Corinebacterium spp., Bacillus spp., Bacillus anthracis, Clostridium spp., Spherophorus spp., Trychophyton spp., Microsporum spp., Micobacterium spp., Vibrio spp., Cryptosporidia spp., Microsporidia spp., Listeria monocytogenes, Lawsonia intracellularis, Treponema desynteriae, Enteroccocus spp., Heamophylus spp., Campylobacter spp., Chlamydia spp., Brucella spp., and other pathogenic bacterial species.

The therapeutic pharmaceutical compositions of the present invention can also be used against pathogenic fungi, such as, for example: Absidia spp., Ajellomyces spp., Arthroderma spp., Aspergillus spp., Blastomyces spp., Candida spp., Cladophialophora spp., Coccidioides spp., Cryptococcus spp., Cunninghamella spp., Epidermophyton spp., Exophiala spp., Filobasidiella spp., Fonsecaea spp., Fusarium spp., Geotrichum spp., Histoplasma spp., Hortaea spp., Issatschenkia spp., Madurella spp., Malassezia spp., Microsporum spp., Mucor spp., Nectria spp., Paecilomyces spp., Paracoccidioides spp., Penicillium spp., Pichia spp., Pneumocystis spp., Pseudallescheria spp., Rhizopus spp., Rhodotorula spp., Scedosporium spp., Schizophyllum spp., Sporothrix spp., Trichophyton spp., Trichosporon spp and others.

The therapeutic pharmaceutical compositions of the present invention can also be used to treat pathogenic protozoa, such as, for example: Cyclospora spp., Cryptosporidium spp., Microsporidium spp., Endamoeba histolytica, Endamoeba hartmanii, Dientamoeba fragilis, Giardia lamblia, Balantidium coli, and Blastocystis hominis as well as some parasitic infections, such as, for example, helminths (nematodes).

Examples of the types of illnesses caused by microbial infections that can be treated in subjects using the therapeutic compositions of the invention include internal infections, such as infections of the gastrointestinal tract, skin, lungs (i.e., pneumonia), kidneys, joints, throat, muscles, and organs, such as the tonsils, sepsis, otitis, sinusitis, conjunctivitis, mastitis, metritis, gastro-enteritis caused by bacteria, fungi, or protozoa, pleuritis, peritonitis, tendonitis, and wounds infected by bacteria. External infections can also be treated, such as dermatitis and boils, also known as abscesses and furuncles, flegmonas and dermatitis. In addition, opportunistic infections seen typically in cases of general poor health, age, or in immunologically compromised individuals, that are caused by usually non-pathogenic microorganisms, may also be treated with embodiments of the invention. In addition some parasitic infections such as helminthiasis (nematodes) may be treated by the therapeutic compositions of the invention.

X. Pilot Therapeutic Study Carvomenthol (V) In Humans

A. Volunteer Groups based on Symptoms of Diarrhea

A formulation of the present invention (carvomenthol) made by reacting equimolar amounts organic carvacrol and menthol was administered orally to human subjects (volunteers) to establish that the preliminary preferred formulation functioned to cure gastrointestinal microbial infections, associated with diarrhea. Common enteric bacteria that cause infectious diarrhea include, for example: Escherichia coli, Campylobacter jejuni, Plesomonas shigelloides, Aeromonas spp, Salmonella spp, Vibrio spp, and Shigella spp. Fungal species that are often associated with intestinal mycosis include, among others, Candida albicans, Candida pseudotropicalis, Geotrichum candidum, and Trichosporon spp. Often these fungal infections are associated with elderly or immunocompromised people. Protozoan species, such as, for example, Entamoeba histolytica, Giardia duodenalis, Cryptosporidium parvum, and Cyclospora cayetanensis can also cause forms of diarrhea.

A capsule comprising with 500 mg of the active ingredient, carvomenthol was administered as an oral formulation. Two 500 mg capsules were administered every 8 or 12 hours. Treatment was designed so that the active ingredient was released throughout the gastrointestinal tract and targeted all parts of the intestines. The patients were all adult volunteers. The testing took place in the United States and most volunteers were farmers, sales agents, and people with a connection to similar products used to treat animals. People elected to be part of the test due to the effectiveness and also to lack of toxicity of similar products in animals. A total of 105 people were included in this study. They were divided into categories according to their symptoms.

1. Acute diarrhea

2. Traveling diarrhea

3. Sub-acute diarrhea

4. Chronic diarrhea

5. Food poisoning diarrhea

As used herein, “successful recovery” means that the subject had improved clinical symptoms. For example, a normalization of body temperature and absence of gastrointestinal distress, i.e., diarrhea. In addition, subjects who took the oral form of the present invention were advised to eat fresh yogurt one hour after treatment as a probiotic to replenish the normal intestinal flora. This led to improved subject homeostasis.

B. Experimental treatments with the preliminary formulation of the invention containing Carvomenthol

1. Experimental Group 1: Acute Diarrhea

Group 1: Acute diarrhea is often caused by bacterial infections, particularly Escherichia coli infection resulting from contact with different strains of bacteria from new sources of food and water.

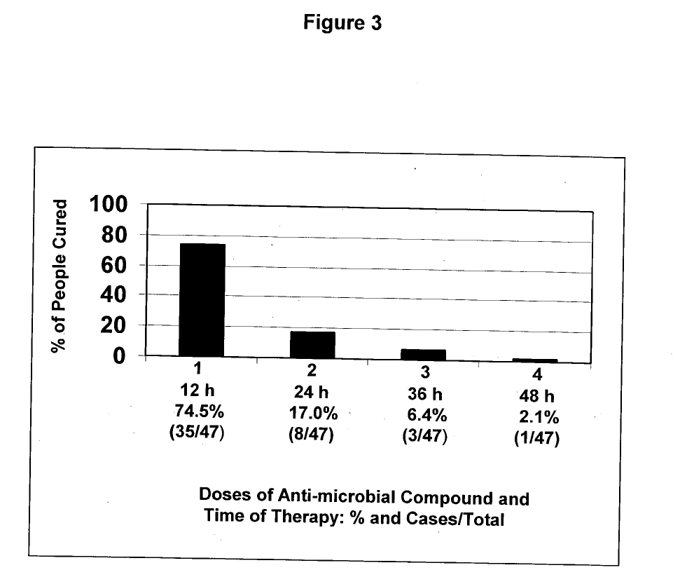

A total of 47 volunteers with acute diarrhea were treated with two 500 mg capsules of the oral formulation administered every 12 hours (FIG. 3). In all cases the diarrhea was cured. In a large number (35) of subjects all symptoms were resolved after a single treatment of the oral formulation. Of the 47 cases, 35 were cured after one, eight required two treatments, three required three treatments, and a single person required four treatments to resolve all symptoms.

2. Experimental Group 2: Traveling Diarrhea

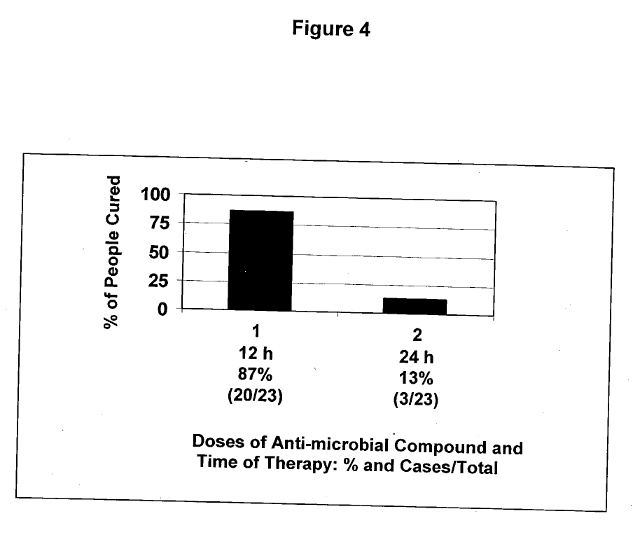

A total of 23 people who had traveled to a new geographical location within the United States or to Mexico were included in this group. Frequently, traveling exposes individuals to new bacterial species and strains, primarily from water, fresh produce, and other food. This exposure to new bacteria may upset the natural intestinal flora and result in “traveling diarrhea.” Subjects for this study were treated with two 500 mg capsules administered every 12 hours (FIG. 4). In 20 cases, a single treatment of the oral formulation of the present invention was required to eliminate traveling diarrhea. In three additional cases, two treatments were necessary to resolve all symptoms.

3. Experimental Group 3: Sub-Acute Diarrhea

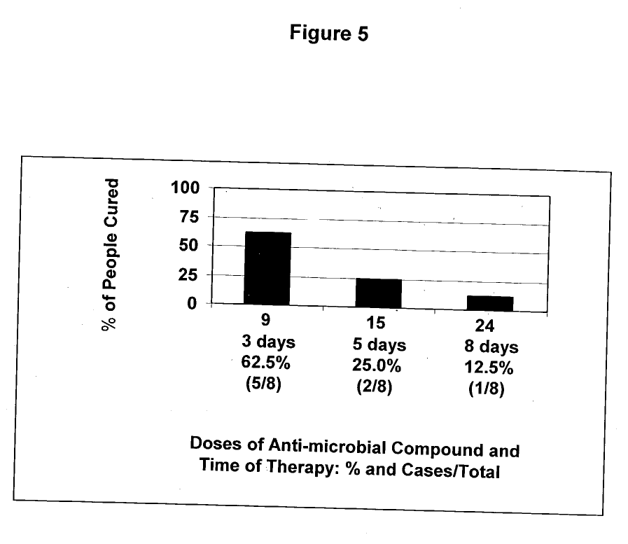

Sub-acute diarrhea is diarrhea that has persisted for 10 days or longer. The eight subjects in this group were 65 years of age or older and had diarrhea that had been unsuccessfully treated with conventional antibiotics (FIG. 5). Two 500 mg capsules of the oral formulation were administered every 8 hours. In five cases, the sub-acute diarrhea was eliminated after 9 treatments over three days. In two cases, 15 doses over five days were required to rid the subject of sub-acute diarrhea. One subject required 24 treatments over 8 days to become symptom free. In all cases the people responded to the therapy and were cured of the infection causing the diarrhea.

4. Experimental Group 4: Chronic Diarrhea

Most of the subjects in this category had a mixed infection of fungi and bacteria in the small and large intestines. Chronic diarrhea is classified as diarrhea that has persisted for two weeks or more. It is often caused from one or more pathogenic organisms. According to laboratory testing, the pathogenic fungi was Candida albicans in the group of five volunteers. The contributing bacterial infection was undiagnosed. In all cases different antifungal therapy was attempted unsuccessfully.

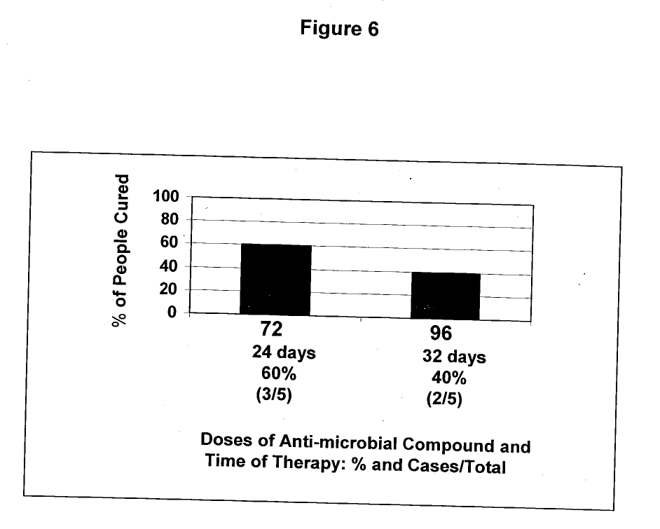

In the five subjects, the symptoms had persisted for 120 days or more. Two 500 mg capsules containing the oral formulation were administered every 8 hours (FIG. 6). Three of the subjects treated in this group responded after 10 days or 30 treatments and were symptom free thereafter. Two subjects required a longer treatment regime of 32 days or 96 doses before they were symptom free. These two particular people had suffered from chronic diarrhea symptoms for more than a year before the current treatment.

5. Experimental Group 5: Food Poisoning Diarrhea

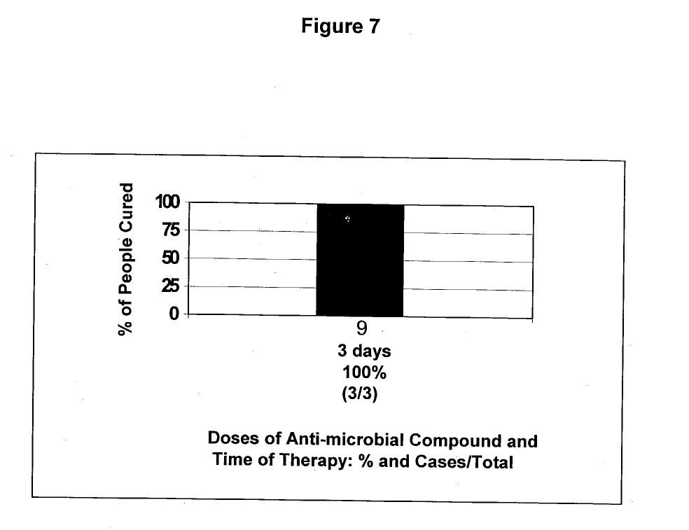

Food poisoning diarrhea is associated with bacteria that produce toxins and include bacteria species such as, for example, Salmonella spp., Staphylococcus aureus, Bacillus cereus and Clostridium perfringens. Symptoms usually manifest within 6 to 12 hours. The three subjects in this group complained of abdominal pain with profuse diarrhea and fever within 12 hours of consuming food. Treatments with an oral formulation of the present invention began immediately and consisted of two 500 mg capsules administered every 8 hours (FIG. 7). In all three cases, after three treatments over a 24 hour period the fever and abdominal pain were eliminated. The diarrhea was still present on the second day, but all symptoms were eliminated by the third day of treatment. In these cases, 3 days of treatment or 9 doses were necessary to resolve all of the food poisoning symptoms. All of these subjects were encouraged to continue treatment for two additional days and were also advised to eat fresh yogurt one hour after treatment as a probiotic to replenish the normal intestinal flora.

XI. Pilot Therapeutic Study Menthocarvol (VI) In Humans

In one embodiment menthocarvol was made by reacting carvacrol with hydrogen under a nitrogen atmosphere in the presence of platinum, as a catalyst, to form menthocarvol. The menthocarvol was administered orally as capsules and topically as ointments to human volunteers to establish that the preliminary preferred formulation functioned to cure specific disease conditions.

A. Effect Of Menthocarvol On Patients With Diarrhea

Four human subjects were selected for this study with menthocarvol. In this study, the diarrhea symptoms were likely, from either acute bacterial infectous causes or food poisoning. The menthocarvol was administered orally in the form of capsules. Each 500 mg capsule contained 35 mg of the active ingredient. The therapy for each subject was usually oral administration of two 500 mg capsules.

1. Case 1: Male, 38 years of age

The subject reported symptoms of severe diarrhea, fever, abdominal cramps, and dizziness that began one hour before treatment was administered. The subject reportedly ate poultry 12 h before the diarrhea symptoms Manifested. The suspected cause of the acute diarrhea was bacterial salmonellosis. The therapy consisted of one oral dose of two 500 mg menthocarvol capsules. The result of the treatment was that all diarrhea-associated symptoms disappeared a few hours after the administration of menthocarvol. Oral rehydration was also given to replenish lost fluids. A complete recovery was observed in three days.

2. Case 2: Female, 29 years of age

The subject reported symptoms consistent with mild food poisoning diarrhea. The patient noted that nausea and vomiting and started approximately 12 h before treatment with menthocarvol. No fever or abdominal cramps were evident. The therapy was oral administration of two 500 mg capsules of menthocarvol. The nausea and vomiting decreased and disappeared within several hours of treatment with the menthocarvol. Oral rehydration was also given to replenish lost fluids. The patient had a complete recovery in two days.

3. Case 3: Male, 43 years of age

The patient complained of symptoms of mild diarrhea that had been present for two days. He had no fever, vomiting, or abdominal cramps. The suspected cause was mild acute bacterial diarrhea. The therapy was oral administration of two 500 mg capsules of menthocarvol (1000 mg). The results of treatment were that all symptoms disappeared in six hours. Oral rehydration was also given to replenish lost fluids. Complete recovery was observed in 24 hours.

4. Case: Male, 36 years of age

The patient reported symptoms consistent with acute diarrhea. These consisted of nausea, vomiting and abdominal cramps. The treatment was oral administration of two 500 mg capsules of menthocarvol, which resulted in cessation of all symptoms within 24 hours. Oral rehydration was also given to replenish lost fluids. Complete recovery was observed in two days.

In each of the four patients the diarrhea was cured quickly after treatment with menthocarvol. None of the patients reported unpleasant or adverse side effects. The menthocarvol proved to be effective in all of the cases with all symptoms being resolved shortly after treatment.

B. Effect of Menthocarvol on Chronic Fatigue

The presence of bacteria (Streptococcus aureus), parasites (entamoeba gingivalis, or trichomonas tenax) and/or fungi (Candida, or Aspergillus species) in the mouth, nose, or sinus cavities is often accompanied by a history of generalized malaise, fatigue, and frequent headaches, frequently referred to as general fatigue syndrome. Laboratory testing for pathogens is usually negative. People suffering from fatigue syndrome typically function at a substantially lower level of activity than they were capable of before the onset of fatigue symptoms. Fatigue is not improved by bed rest and may be exacerbated by physical or mental activity. Moreover, the presence of some microorganisms in the stomach (i.e. Helicobacter pylori) and bowels, especially in diverticulas (outpouchings of the intestinal mucosa), is often accompanied by malaise and headache.

In the United States, colonic diverticula occurs in approximately 50% of individuals older than 60 years of age. Treatment with menthocarvol of volunteers suffering from this chronic fatigue syndrome was conducted. The menthocarvol therapy was administered in an oral dose of a single 250 mg capsule (17.5 mg active ingredient) or 500 mg capsule (35 mg active ingredient). Two of the subjects, who had localized pain in an extremity, were treated topically with menthocarvol in an ointment (3% active ingredient in lanolin oil). A total of six active individuals complaining of fatigue syndrome participated in this study. The symptoms and results of treatment for the six patients are listed below:

1. Case 1: Male, 36 years of age, and an active runner

The subject complained of symptoms of impaired concentration, headache, unrefreshing sleep, and post-exertional malaise, lasting more than 24 hours. Treatment was an oral administration of a 250 mg capsule of menthocarvol after breakfast for five days. After the treatment the symptoms of malaise and unrefreshing sleep disappeared. No adverse side effects were reported.

2. Case 2: Female, 29 years of age, and an active runner

The subject complained of symptoms of fatigue accompanied by a sore throat, loss of appetite, muscle pain, and multi-joint pain. There was no obvious swelling of the joints. Treatment was an oral administration of a 250 mg capsule of menthocarvol after breakfast for ten days. The sore throat and pain disappeared after five days of treatment as well as a restoration of normal appetite. The woman reported improved running workouts and an improved overall healthful feeling.

3. Case 3: Male, 38 years of age, and a tennis player

The subject complained of symptoms of severe unexplained fatigue and headache that was not associated with any known medical problem. Treatment was oral administration of a 250 mg capsule of menthocarvol after breakfast for five days. After the treatment the symptoms of fatigue and headache were relieved. No adverse side effects were reported.

4. Case 4: Male 55 years of age, and a marathon runner

The subject complained of symptoms of widespread musculoskeletal pain with disturbed sleep accompanied by morning stiffness. Treatment was oral administration of a 500 mg capsule of menthocarvol after breakfast for five days. After the treatment the fatigue and muscle soreness disappeared. No adverse side affects were reported.

5. Case 5: Male 64 years of age, and a regular runner

The subject complained of symptoms of pain and leg cramps in the during prolonged running and exercise. Therapy was to apply a topical menthocarvol cream (3% active ingredient in lanolin oil) to the sore leg areas before exercise over ten days. The signs of leg and foot muscle fatigue disappeared after the tenth treatment.

6. Case 6: Male 49 years of age, and a runner

The subject complained of pain and swelling in both of Achille's tendons after prolonged running, especially when running hills. Treatment included application of a menthocarvol-containing cream (3% active ingredient in lanolin oil) to the affected areas before running. The pain and swelling was significantly reduced after six applications.

In all cases menthocarvol has demonstrated positive effects on the fatigue and pain of the people in this study. No subjects complained of unpleasant or adverse side effects from the treatment. It is thus believed that the oral and topical formulations are safe and effective.

C. Effect of Menthocarvol on Periodontal Disease

Gum infection represents a common form of oral disease known as periodontitis. Bacterial plaque in the mouth can accumulate in hard-to-clean areas along the gum line and between the teeth, causing infection, inflammation, and damage to the gum tissue, resulting in the gums pulling away from the teeth allowing gaps or pockets to form. These areas are havens for oral bacteria to thrive and are eventually filled with more bacterial plaque, tartar (calculus), pus and food debris. This type of infection is often painless, however after a prolonged time without treatment the bone support anchoring the teeth also begins to regress away from the teeth and gums. Periodontitis is the leading cause of tooth loss in adults.

Most periodontal disease arises from, or is aggravated by, accumulation of plaque, and is associated particularly with anaerobic bacterial species such as, for example, Porphyromonas gingivalis, Bacteroides forsythus, and Actinobacillus actinomycetemcomitans.

A study on three subjects was performed to test the effectiveness of orally applied ointments containing menthocarvol to treat periodontal disease in humans. The preliminary formulation used was first was a 3% solution of menthocarvol followed by treatment with a 0.6% solution, administered twice a week for three months. The treatment was given with a syringe and applied orally to the affected areas between the teeth at the gum line. Patients reported that the formulation had a pleasant taste and smell. The application of the 3% solution caused a temporary tingling sensation on the gums that was slightly more pronounced on areas of the tongue that also contacted the menthocarvol solution. Nevertheless, there was no observable necrotic effect of this compound on the epithelial cells lining the mouth cavity. This phenomenon disappeared in approximately two hours. This side effect was not observed during application of the 0.6% solution, but also the therapeutic effect was not as pronounced, compared to the 3% solution over the treatment period. All three of the patients responded to the application of the 3% menthocarvol solution and a marked improvement in the inflammation of their gums was observed. The same people showed some improvement when treated solely with the 0.6% menthocarvol solution, but the reduction of infection and observable periodontal disease was not as significant as that observed with the 3% menthocarvol treatment.

XII. Working Examples

A. Carvomenthol Capsules and Tablets

The formulation of the preferred embodiment highlighted in section V contained the novel antimicrobial compound carvomenthol, which was manufactured into capsules and tablets. The formula for these are shown below:

| | |
|---|---|
| Ingredient | weight % |

Capsule

| | |
|---|---|
| Menthol | 2.5 |
| Carvacrol | 3.5 |
| Silicon dioxide | 6.0 |
| Starch | 35.0 |
| Lactose | 53.0 |

Tablet

| | |
|---|---|
| Menthol | 2.5 |
| Carvacrol | 3.5 |
| Silicon dioxide | 4.0 |
| Sorbitol | 7.0 |
| Colloid | 20.0 |
| Dextrose | 63.0 |

Each capsule was filled with the complete active ingredient carvomenthol compounded with the other ingredients. These were either 300 mg or 500 mg sized capsules. In the method of manufacture first the menthol and carvacrol were reacted and then combined with silicon dioxide in a commercial TBI-27 mixing device capable of mixing each component at a 90 degree angle to each other (Techno-Industrie, GmbH, Germany). These ingredients were mixed for about 5 minutes at approximately 1,000 revolutions per minute (rpm). The starch and lactose were then added for capsules, or sorbitol and dextrose for tablets, and the total recipe mixed for an additional 5 minutes in the same device at 1,700 rpm. For capsules, the evenly reacted and compounded antimicrobial compound was subject to trituration and aliquoted into capsule shells with an encapsulating machine (Type TIE-M4 Techno-Industrie, GmbH, Germany). For tablets, the powder mixture is processed through a trough granulator device to produce a therapeutic granule fine enough to make tablets (GA-3C, TBCB, South Korea). The preferred dimensions of the granules were selected to be between 1-3 mm. The compound was then dried in an oven at about 75° C. for one hour. The colloidal material was then added evenly under pressure. Individual tablets were pressed to have weights of 300 mg and 500 mg. The tablets contain colloid to keep the other materials together in the tablet and to aid in dispersion of the active ingredient carvomenthol.

B. Menthocarvol Capsules and Tablets

The formulation of the preferred embodiment highlighted in section VI contained the novel antimicrobial compound menthocarvol, which was manufactured into both capsules and tablets. The formula and method for these are as follows.

| | |
|---|---|
| Ingredient | weight % |

Capsule

| | |
|---|---|
| Menthocarvol | 5.0 |
| Silicon Dioxide | 6.0 |
| Sorbitol | 10.0 |
| Fructose | 30.0 |
| Dextrose | 49.0 |

Tablet

| | |
|---|---|
| Menthocarvol | 5.0 |
| Silicon Dioxide | 6.0 |
| Sorbitol | 10.0 |
| Colloid | 20.0 |
| Fructose | 25.0 |
| Dextrose | 34.0 |

In the method of manufacture, first the menthol and carvacrol were reacted and then combined with the silicon dioxide in a commercial TBI-27 mixing device capable of mixing each component at a 90 degree angle to each other (Techno-Industrie, GmbH, Germany). These ingredients were mixed slowly at a maximum speed of about 500 revolutions per minute (rpm). This was done for about 5-20 minutes until the mixture was homogenous. The sorbitol, fructose, and dextrose were then added and the total recipe mixed for about an additional 5 minutes at 1,000 rpm, followed by an additional mixing for about 5 minutes at 1,700 rpm. For capsules, the powder was subject to trituration and aliquoted into capsule shells with an encapsulating machine (Type TIE-M4 Techno-Industrie, GmbH, Germany). For tablets, the powder mixture was processed through a trough granulator device to produce a therapeutic granule fine enough to make tablets (GA-3C, TBCB, South Korea). The preferred dimensions of the granules were selected to be between 1-3 mm. The compound was then dried in an oven at about 75° C. for one hour. The colloidal material was then added evenly under pressure. Individual tablets were pressed to have weights of 300 mg and 500 mg.

C. Menthocarvol Topical Ointment

The formulation of the preferred embodiment highlighted in section VI contained the novel antimicrobial compound carvomenthol, which was manufactured into tablets.

| Ingredient | weight % |
|---|---|
| Menthocarvol | 3.0-5.0 |
| Emulsifier | 20.0 |
| Lanalol | 25.0 |
| Water | 50.0-52.0 |

The active ingredient menthocarvol was made from carvacrol and hydrogen under a nitrogen atmosphere using platinum as a catalyst. Heat and was applied and stirring maintained throughout the reaction which was performed in a pressure chamber vessel. The temperature was allowed to reach 170° C. when hydrogen was allowed to flow into the reaction under approximately 3.2 bars of pressure. The reaction was allowed to proceed for about 6 h and then cooled to 25° C. The catalyst platinum was precipitated and the reactor content was transferred to a filtration device. Upon separation of the solid phase from the oil fluid part analysis of the oily fluid was performed. The analysis was done using gas mass chromatography (GMC) on a HP-GL-1900 C GCD apparatus using standard settings needed to evaluate the reaction. The analysis confirmed that menthocarvol was produced with a yield of approximately 85% based on content.

The menthocarvol was first mixed with the lanolin at about 1,200 rpm for 5 minutes. The emulsifier (sylicogel) and water were added and the contents mixed for an additional 10 minutes at about 1,200 rpm. The contents were further mixed for 15 minutes at about 1,800 rpm to ensure the therapeutic menthorcarvol compound was a stable homogenous ointment.

D. Oral Menthocarvol Periodontal Solution

The oral periodontal solution was made from the following formulation:

| Ingredient | weight % |
|---|---|
| Menthocarvol | 3.0 |
| Emulsifier | 5.0 |
| Propylene Glycol | 92.0 |

The active ingredient menthocarvol was made as in working example 3 and was combined with the emulsifier (sylicogel) and the propylene glycol by mixing for about 5-10 minutes in the TBI-27 mixer (Techno-Industrie, GmbH, Germany) at 1,200 rpm. The 3% formulation was diluted with propylene glycol to 0.6% of the menthocarvol to form this more diluted form of the oral therapeutic.

While the invention has been described by means of specific embodiments and applications thereof, it is understood that numerous modifications and variations could be made thereto by those skilled in the art without departing from the spirit and scope of the invention. It is therefore to be understood that within the scope of the claims, the invention may be practiced otherwise than as specifically described.

We claim:

1. A therapeutic composition for treating a microbial infection in a subject, said composition comprising:
(a) an antimicrobial compound, wherein said antimicrobial compound further comprises an organic phenolic compound combined with an essential oil alcohol; and
(b) a pharmaceutically acceptable carrier.

2. The therapeutic composition of claim 1, wherein said antimicrobial compound is carvomenthol.

3. The therapeutic composition of claim 1, wherein said antimicrobial compound is thymomenthol.

4. The therapeutic composition of claim 1, wherein said organic phenolic compound and said essential oil alcohol are combined via a chemical reaction.

5. The therapeutic composition of claim 1, wherein said organic phenolic compound and said essential oil alcohol are combined in a mixture.

6. The therapeutic composition of claim 1 further comprising an additive selected from the group consisting of vitamins, minerals, amino acids, fats, oils, and combinations thereof.

7. The therapeutic composition of claim 1, wherein said organic phenolic compound is selected from the group consisting of carvacrol, synthetic carvacrol, thymol, synthetic thymol, hypericine, natural tannins, natural polyphenols, and combinations thereof.

8. The therapeutic composition of claim 1, wherein said essential oil alcohol is selected from the group consisting of hexahydrothymol, hexahydrocarvacrol, and combinations thereof.

9. The therapeutic composition of claim 1, wherein said antimicrobial compound comprises up to about 10% of the total weight percentage of the antimicrobial compound.

10. The therapeutic composition of claim 1, wherein said antimicrobial compound comprises about 85-95% of a first antimicrobial compound and about 5-15% of a second antimicrobial compound.

11. The therapeutic composition of claim 1, wherein said microbial infection is selected from the group consisting of a bacterial infection, a yeast infection, a fungal infection, a protozoan infection, a parasitic infection, and combinations thereof.

12. The therapeutic composition of claim 11, wherein said microbial infection is an opportunistic infection wherein a primary defect is selected from the group consisting of a viral infection, stress, general poor health, age, and a compromised immune system.

13. A therapeutic composition for treating a microbial infection in a subject, said composition comprising:
(a) an antimicrobial compound, wherein said antimicrobial compound comprises hexahydrocarvacrol; and
(b) a pharmaceutically acceptable carrier.

14. The therapeutic composition of claim 13 further comprising an additive selected from the group consisting of vitamins, minerals, amino acids, fats, oils, and combinations thereof.

15. The therapeutic composition of claim 13, wherein said antimicrobial compound comprises up to about 10% of the total weight percentage of the antimicrobial compound.

16. The therapeutic composition of claim 13, wherein said antimicrobial compound comprises about 85-95% of a first antimicrobial compound and about 5-15% of a second antimicrobial compound.

17. The therapeutic composition of claim 13, wherein said microbial infection is selected from the group consisting of a bacterial infection, a yeast infection, a fungal infection, a protozoan infection, a parasitic infection, and combinations thereof.

18. The therapeutic composition of claim 13, wherein said microbial infection is an opportunistic infection wherein a primary defect is selected from the group consisting of a viral infection, stress, general poor health, age, and a compromised immune system.

19. A method for treating a microbial infection in a subject comprising administering to the subject in need thereof an antimicrobial compound, wherein said antimicrobial compound compromises an organic phenolic compound combined with an essential oil alcohol and a pharmaceutically acceptable carrier.

20. The method of claim 19, wherein said antimicrobial compound is carvomenthol.

21. The method of claim 19, wherein said antimicrobial compound is thymomenthol.

22. The method of claim 19, wherein said organic phenolic compound and said essential oil alcohol are combined in a chemical reaction.

23. The method of claim 19, wherein said organic phenolic compound and said essential oil alcohol are combined in a mixture.

24. The method of claim 19 further comprising an additive selected from the group consisting of vitamins, minerals, amino acids, fats, oils, and combinations thereof.

25. The method of claim 19, wherein said organic phenolic compound is selected from the group consisting of carvacrol, synthetic carvacrol, thymol, synthetic thymol, hypericine, natural tannins, natural polyphenols, and combinations thereof.

26. The method of claim 19, wherein said essential oil alcohol is selected from the group consisting of hexahydrothymol, hexahydrocarvacrol, and combinations thereof.

27. The method of claim 19, wherein said antimicrobial compound comprises up to about 10% of the total weight percentage of the antimicrobial compound.

28. The method of claim 19, wherein said antimicrobial compound comprises about 85-95% of a first antimicrobial compound and about 5-15% of a second antimicrobial compound.

29. The method of claim 19, wherein said microbial infection is selected from the group consisting of a bacterial infection, a yeast infection, a fungal infection, a protozoan infection, a parasitic infection, and combinations thereof.

30. The method of claim 19, wherein said microbial infection is an opportunistic infection wherein a primary defect is selected from the group consisting of a viral infection, stress, general poor health, age, and a compromised immune system.

31. A method for treating a microbial infection in a subject comprising administering to a subject in need thereof an antimicrobial compound, wherein said antimicrobial compound comprises hexahydrocarvacrol and a pharmaceutically acceptable carrier.

32. The method of claim 31 further comprising an additive selected from the group consisting of vitamins, minerals, amino acids, fats, oils, and combinations thereof.

33. The method of claim 31, wherein said antimicrobial compound comprises up to about 10% of the total weight percentage of the antimicrobial compound.

34. The method of claim 31, wherein said antimicrobial compound comprises about 85-95% of a first antimicrobial compound and about 5-15% of a second antimicrobial compound.

35. The method of claim 31, wherein said microbial infection is selected from the group consisting of a bacterial infection, a yeast infection, a fungal infection, a protozoan infection, a parasitic infection, and combinations thereof.

36. The method of claim 35, wherein said microbial infection is an opportunistic infection where a primary defect is selected from the group consisting of a viral infection, stress, general poor health, age, and a compromised immune system.
